(19) United States
(12) Patent Application Publication
(10) Pub. No.: US 2002/0183937 A1
(43) Pub. Date: Dec. 5, 2002

(54) Method for the generation of proteins with new enzymatic function

(76) Inventors: Stephen Mayo, Pasadena, CA (US); Daniel N. Bolon, Pasadena, CA (US)

Correspondence Address: ROBIN M. SILVA, ESQ., FLEHR HOHBACH TEST ALBRITTON & HERBERT LLP, Four Embarcadero Center - Suite 3400, San Francisco, CA, 94111-4187, US

(21) Appl. No.: 10/074,679

(22) Filed: Feb. 11, 2002

Related U.S. Application Data
(60) Provisional application No. 60/267,602, filed on Feb. 9, 2001.

(51) Int. Cl. G06F 19/00; G01N 33/48; G01N 33/50

(52) U.S. Cl. 702/19

(57) ABSTRACT

The invention relates to the use of a variety of computational methods for generating enzyme-like protein catalysts. Specifically, computational methods are used to insert active site domains, including catalytic domains and binding domains, into a protein scaffold and optimize surrounding amino acids for interaction with the active site domain.

### FIELD OF THE INVENTION

The invention relates to the use of a variety of computational methods for generating enzyme-like protein catalysts. Specifically, computational methods are used to insert active site domains, including catalytic domains and binding domains, into a protein scaffold and optimize surrounding amino acids for interaction with the active site domain.

### BACKGROUND OF THE INVENTION

The ability to design an enzyme to perform a given chemical reaction has considerable practical application for industry and medicine, particularly for the synthesis of pharmaceuticals (Liese, A. & Filho, M. V., (1999) Curr Opin Biotechnol, 10: 595-603). Although, significant progress has been made at enhancing the catalytic properties of existing enzymes through directed evolution (see Arnold, F. H., & Volkov, A. A., (1999) Curr Opin Chem Biol, 3: 54-59), the design of proteins with novel catalytic properties has met with limited success (Corey, M. J. & Corey, E., (1996) Proc Natl Acad Sci USA, 93: 11428-11434; Petsko, G. A., (2000) Nature, 403: 606-607).

Design strategies used to generate proteins with novel catalytic functions have used transition state analogs as haptens to elicit catalytic antibodies or have altered existing active site residues to generate proteins that catalyze new reactions. Although transition state analogs have been successfully used as haptens to generate catalytic antibodies (Hilver, D., (2000) Annu Rev Biochem, 69: 751-793; and, Wagner, J., et al. (1995) Science, 270: 1797-1800, this approach does not permit the efficient selection of catalytic side chains and transition state stabilization in the same molecule. In addition, because the relationship between the general backbone fold of an enzyme and its catalytic properties is not well understood, this complicates the design of catalytic antibodies in which the active site is restricted to the antibody fold.

Other approaches include altering existing active site residues to generate novel protein catalysts. For example, cyclophilin, a cis-trans isomerase of X-Pro peptide bonds was engineered into an endopeptidase by grafting a triad a catalytic residues commonly found in serine proteases at the binding cleft (Quemeneur, E., et al., (1998) Nature, 391: 301-304). In a more complicated design effort, indoleglycerol-phosphate synthase was converted into phosphoribosylanthranilate isomerase (Altamirano, M. M., et al., (2000) Nature, 403:617-622). This approach capitalized on the fact that the naturally occurring versions of the these enzymes share a similar fold. Thus, based on a comparison of the crystal structures of both natural enzymes, loops were altered in the synthase to resemble the target isomerase, followed by directed evolution.

Although rational non-computational approaches have been successfully used to alter substrate specificity or catalytic mechanism, these approaches are of limited use for enzyme design strategies utilizing a starting protein scaffold devoid of substrate binding and catalytic activities because of the requirement for introducing residues that can convert an inactive scaffold protein into a protein with catalytic activity. One way to overcome the problem of introducing active site residues into an inactive scaffold protein is to use computational modeling methods for the design of proteins with novel catalytic properties.

Computational modeling methods have been used to introduce metal binding sites into proteins (Hellinga, H. W. & Richards, F. M., (1991) J Mol Biol, 222: 763-785; Robertson, D. E., et al., (1994) Nature, 368: 425-432; and, Klemba, M., et al., (1995) Nat Struct Biol, 2:368-373). By leaving one of the primary coordination spheres of the metal un-ligated by the protein, nascent metalloenzymes with a variety of oxygen redox chemistries have been generated OPinto, A. L., et al., (1997) Proc Natl Acad Sci USA, 94:5562-5567; and, Benson, D. E., et al., (2000) Proc Natl Acad Sci USA, 97: 6292-6297).

However, the ability of these “designed metalloenzymes” to specifically react with more complicated organic molecules has not been demonstrated.

Accordingly, it is an object of the invention to use computational methods to generate proteins with novel catalytic and/or binding properties. By combining basic principles of enzymatic catalysis, including proximity and orientation of substrate molecules, transition state stabilization, acid-base catalysis, and covalent catalysis (Fersht, A., Enzyme Structure and Mechanism, Freeman, New York, 1985) with computational modeling methods such as protein design algorithms, (U.S. Pat. Nos. 6,188,965; 6,269,312; Dahiyat, B. L I & Mayo, S. L., (1997) Science, 278:82-87; Harbury, P. B., et al., (1998) Science, 282:1462-1467; Street, A. G. & Mayo, S. L., (1999) Structure Fold Des, 7:R105-109; Raha, K., et al. (2000) Protein Sci., 9:1106-1119) and force field calculations (Gordon, D. B., (1999) Curr Opin Struct Biol, 9:509-513) novel proteins can be made and evaluated for enzyme-like activities.

### SUMMARY OF THE INVENTION

In accordance with the objects outlined above, the present invention provides methods executed by a computer under the control of a program, the computer including a memory for storing the program. The method comprising the steps of identifying a suitable protein scaffold lacking a “enzyme-like activity”, imputing the scaffold protein backbone structure with variable residue positions, inserting an “enzyme-like” domain into the scaffold, and applying at least one protein design cycle to generate a set of candidate variant proteins with putative enzyme-like activity.

Protein design cycles that may be used to generate variable protein sequences include PDA™, sequence prediction algorithm, and force field calculations. The protein design cycle may include a Dead-End Elimination (DEE) computation. Generally, the analyzing step includes the use of at least one scoring function selected from the group consisting of a van der Waals potential scoring function, a hydrogen bond potential scoring function, an atomic solvation scoring function, a secondary structure propensity scoring function and an electrostatic scoring function. Some or all of the protein sequences from the ordered list may be tested for enzyme-like activity.

In an additional aspect, the invention provides for the synthesis of a plurality of secondary sequences to generate libraries of putative protoenzymes. The libraries may be optionally synthesized and tested, in a variety of ways, including error prone PCR, gene shuffling, etc.

In an additional aspect, the invention provides nucleic acid sequences encoding a protein sequence generated by the present methods, and expression vectors and host cells containing the nucleic acids.

### BRIEF DESCRIPTION OF THE DRAWINGS

FIG. 1 illustrates processing steps associated with a preferred embodiment of the invention. In this embodiment, PDA™ is used to insert high energy state rotamers into a protein scaffold and select amino acids at surrounding positions that interact favorably with the high energy state rotamers to form an active site domain. Sequences containing putative active site domains are selected and tested for enzyme-like activity.

FIG. 2A illustrates nucleophile mediated catalysis of PNPA hydrolysis.

FIG. 2B illustrates the high energy structure used in the computational active site scan. Labeled dihedral angles were varied as indicated in order to generate the set of high energy state rotamers used in the design calculations.

FIG. 3 illustrates the computational design of PZD2. Ribbon diagram (Koradi, R., et al., (1996) J Mol Graph, 14: 51-55; and 29-32.

FIG. 4 illustrates molecular surfaces (Nicholls, A., et al., (1991) Proteins, 11: 281-296) focusing on the active site of PZD2 with substrate atoms (see FIG. 4A) and the corresponding region in the x-ray crystal structure (Katti, S. K., et al., (1990) J Mol Biol, 212: 167-184) of the wild type scaffold (see FIGS. 4B & 4C). An active site cleft is present in the design of PZD2 that is largely filled in the wild type structure. Wild type residues that were mutated to create the active site are shown in FIG. C (F12, L17, and Y70). In the design of PZD2, all side chains were allowed to change rotamers, resulting in a slightly different surface compared to that of the wild type protein.

FIG. 5 illustrates the kinetic model used to analyze the activity of PZD2.

FIG. 6 illustrates velocity versus substrate concentration for the hydrolysis of PNPA by PZD2.

FIG. 7 depicts buffer corrected hydrolysis of PNPA by PZD2 (•), PZD2H17A (□), wild type thioredoxin (▴), and the wild type L17H/D261 (×). Data are shown for high substrate concentration and equivalent low protein concentration.

FIG. 8 illustrates trapping of an acylated intermediate by mass spectrometry: A) PZD2; B) PZD2 reacted with substrate; C) PZD2H17A; and D) PZD2H17A reacted with substrate. A large increase in the population of +42 species occurs upon reaction of PZD2 with substrate indictating the buildup of an acyl-enyzme intermediate. This +42 species is dramatically reduced for PZD2H17A where the designed catalytic histidine was mutated to alanine. A small increase in the population of a +42 species is detected in PZD2H17A upon reaction with substrate and is likely the result of acylation at the single surface exposed histidine at position 6. Consistent with this analysis, a small increase in the population of a double acetylated +84 product is detected upon reaction of PZD2 with substrate. A copper matrix adduct (+63) is present in all spectra (Katti, S. K., et al., (1990) J Mol Biol, 212:167-184).

FIG. 9 depicts a Lineweaver-Burk analysis of PZD2 catalyzed PNPA hydrolysis in the presence (□) and absence (●) of 10 mM PNPG.

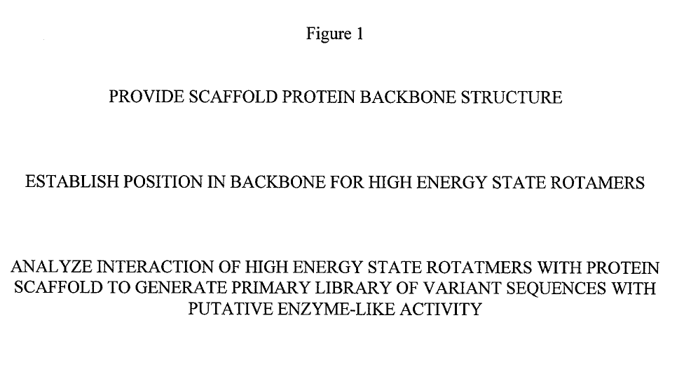

### DETAILED DESCRIPTION OF THE INVENTION

The present invention is directed to computational methods for the design of proteins with novel functions, including catalytic and/or binding activities. An important step in the design of a protein of transition state analog synthesis for catalytic antibody design.

For example, if a protein with serine protease activity is desired an active site domain, e.g., the amino acids conferring activity, comprising one or more catalytic residues, may be inserted into a protein scaffold. Favorable positions for the location of the active site domain may be identified using computational methods to search for positions along the backbone of the protein scaffold where the active site domain and the substrate can be properly positioned such that the desired chemical reaction occurs.

Alternatively, it may be desirable to begin with an active site domain and then use structural homology methods to choose an appropriate protein scaffold for the insertion of the domain. Computational methods may then be applied to determine the best location for the domain and to optimize the amino acids in the surrounding area to accommodate substrate binding and catalysis.

Basic principles of enzymatic catalysis including, but not limited to, proximity and orientation of substrate molecules, transition state stabilization, acid-base catalysis, and covalent catalysis may be used as guidelines for building an active site domain. Computational methods can then be applied to generate proteins with novel catalytic activity, i.e. protozymes. Additional catalysts, as well as optimization of computer generated catalysts may be achieved by combining computational design methods with experimental directed evolution methods.

Accordingly, the present invention provides methods for generating protozymes. By “protozymes” herein is meant proteins with novel enzyme like activities including, but not limited to, catalytic function or ligand binding properties. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992)], generally depending on the method of synthesis. Thus “amino acid”, or “peptide residue”, as used herein means both naturally occurring and synthetic amino acids. For example, homo-phenylalanine, citrulline, and noreleucine are considered amino acids for the purposes of the invention. “Amino acid” also includes imino acid residues such as proline and hydroxyproline. In addition, any amino acid representing a component of the variant proteins of the present invention can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R- or the S-, depending upon its composition and chemical configuration. Such derivatives generally have the property of greatly increased stability, and therefore are advantageous in the formulation of compounds which may have longer in vivo half lives, when administered by oral, intravenous, intramuscular, intraperitoneal, topical, rectal, intraocular, or other routes.

In the preferred embodiment, the amino acids are in the (S) or L-configuration. If non-naturally occurring side chains are used, non-amino acid substituents may be used, for example to prevent or retard in vivo degradations. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U 138 Part 2 Aug. 22, 1999, both of which are expressly incorporated by reference herein.

Aromatic amino acids may be replaced with D- or L-naphylalanine, D- or L-phenylglycine, D- or L-2-thieneylalanine, D- or L-1-, 2-, 3- or 4-pyreneylalanine, D- or L-3-thieneylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylphenylalanine, D- or L-p-methoxybiphenylphenylalanine, D- or L-2-indole(alkyl)alanines, and D- or L-alkylalanines where alkyl may be substituted or unsubstituted methyl, ethyl, propyl, hexyl, butyl, pentyl, isopropyl, iso-butyl, sec-isotyl, iso-pentyl, and non-acidic amino acids of C1-C20.

Acidic amino acids can be substituted with non-carboxylate amino acids while maintaining a negative charge, and derivatives or analogs thereof, such as the non-limiting examples of (phosphono)alanine, glycine, leucine, isoleucine, threonine, or serine; or sulfated (e.g., —SO₃H) threonine, serine, or tyrosine.

Other substitutions may include unnatural hydroxylated amino acids may made by combining “alkyl” with any natural amino acid. The term “alkyl” as used herein refers to a branched or unbranched saturated hydrocarbon group of 1 to 24 carbon atoms, such as methyl, ethyl, n-propyl, isoptopyl, n-butyl, isobutyl, t-butyl, octyl, decyl, tetradecyl, hexadecyl, eicosyl, tetracisyl and the like. Alkyl includes heteroalkyl, with atoms of nitrogen, oxygen and sulfur. Preferred alkyl groups herein contain 1 to 12 carbon atoms. Basic amino acids may be substituted with alkyl groups at any position of the naturally occurring amino acids lysine, arginine, ornithine, citrulline, or (guanidino)-acetic acid, or other (guanidino)alkyl-acetic acids, where “alkyl” is define as above. Nitrile derivatives (e.g., containing the CN-moiety in place of COOH) may also be substituted for asparagine or glutamine, and methionine sulfoxide may be substituted for methionine. Methods of preparation of such peptide derivatives are well known to one skilled in the art.

In addition, any amide linkage in any of the variant polypeptides can be replaced by a ketomethylene moiety. Such derivatives are expected to have the property of increased stability to degradation by enzymes, and therefore possess advantages for the formulation of compounds which may have increased in vivo half lives, as administered by oral, intravenous, intramuscular, intraperitoneal, topical, rectal, intraocular, or other routes.

Additional amino acid modifications of amino acids of variant polypeptides of to the present invention may include the following: Cysteinyl residues may be reacted with alpha-haloacetates (and corresponding amines), such as 2-chloroacetic acid or chloroacetamide, to give carboxymethyl or carboxyamidomethyl derivatives. Cysteinyl residues may also be derivatized by reaction with compounds such as bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-n itrobenzo-2-oxa-1,3-diazole.

Histidyl residues may be derivatized by reaction with compounds such as diethylprocarbonate e.g., at pH 5.5-7.0 because this agent is relatively specific for the histidyl side chain, and para-bromophenacyl bromide may also be used; e.g., where the reaction is preferably performed in 0.1 M sodium cacodylate at pH 6.0.

Lysinyl and amino terminal residues may be reacted with compounds such as succinic or other carboxylic acid anhydrides. Derivatization with these agents is expected to have the effect of reversing the charge of the lysinyl residues.

Other suitable reagents for derivatizing alpha-amino-containing residues include compounds such as imidoesters, e.g., as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin according to known method steps. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues per se is well known, such as for introducing spectral labels into tyrosyl residues by reaction with aromatic diazonium compounds or tetranitromethane.

N-acetylimidizol and tetranitromethane may be used to form O-acetyl tyrosyl species and 3-nitro derivatives, respectively. Carboxyl side groups (aspartyl or glutamyl) may be selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Furthermore aspartyl and glutamyl residues may be converted to asparaginyl and glutaminyl residues by reaction with ammonium ions.

Glutaminyl and asparaginyl residues may be frequently deamidated to the corresponding glutamyl and aspartyl residues. Alternatively, these residues may be deamidated under mildly acidic conditions. Either form of these residues falls within the scope of the present invention.

The scaffold protein may be any protein for which a three dimensional structure is known or can be generated; that is, for which there are three dimensional coordinates for each atom of the protein. Generally this can be determined using X-ray crystallographic techniques, NMR techniques, de novo modeling, homology modeling, etc. In general, if X-ray structures are used, structures at 2 Å resolution or better are preferred, but not required.

The scaffold proteins may be from any organism, including prokaryotes, eukaryotes, and viruses with proteins from bacteria, fungi, extremeophiles such as the archebacteria, insects, fish, animals (particularly mammals and particularly human) and birds all possible.

Accordingly, by “scaffold protein” herein is meant a protein that can be computationally modeled to incorporate a novel catalytic function or property. As will be appreciated by those in the art, any number of scaffold proteins find use in the present invention. Specifically included within the definition of “protein” are fragments and domains of known proteins, including functional domains such as enzymatic domains, binding domains, etc., and smaller fragments, such as turns, loops, etc. That is, portions of proteins may be used as well. In addition, “protein” as used herein includes proteins, oligopeptides and peptides. In addition, protein variants, i.e. non-naturally occurring protein analog structures, may be used.

Suitable proteins include, but are not limited to: 1) proteins that are thermodynamically stable and are essentially catalytically inert with respect to the desired enzymatic activity (or, in the case of protozymes that function as binding partners for ligands, the scaffold lacks the binding activity); 2) proteins that are thermodynamically stable and essentially lack catalytic activity; and, 3) proteins that are thermodynamically stable, essentially lack catalytic activity, and do not elicit any therapeutically significant effects, i.e., such as an immune response, when introduced into a patient. A “patient” for the purposes of the present invention includes both humans and other animals. Generally, high thermodynamic stability suggests the protein can tolerate the destabilizing mutations that are required to build an active site (Hellinga, H. W., et al., (1992) Biochemistry, 31: 11203-11209).

In a preferred embodiment, the protein chosen as the scaffold is thermodynamically stable and essentially catalytically inert with respect to the desired enzymatic activity.

In a preferred embodiment, the protein chosen as the scaffold is thermodynamically stable and essentially lacks catalytic activity.

In a preferred embodiment, the protein chosen as the scaffold is thermodynamically stable, essentially lacks catalytic activity and does not elicit any therapeutically significant effect upon introduction into a patient.

Suitable scaffolds include thioredoxin (Holmgren, A., (1985) Annu Rev Biochem, 237-271), human serum albumin, non immunogenic soluble proteins, such as Zn-alpha2-glycoprotein (Sanchez, L. M., (1997) Proc. Natl. Acad. Sci., 94:4626-4630; Sanchez, L. M., et al., (1999) Science, 283:1914-1919; both of which are hereby expressly incorporated by reference), immunoglobulin G, fibronectin derivatives, and other thermodynamically stable proteins that have a free energy of unfolding greater than 2 kcal per mole, etc.

Once a suitable scaffold has been chosen an active site domain is inserted. By “active site domain” herein is meant a domain that has enzyme-like activity, including catalytic activity or ligand binding activity. By “enzyme-like activity” or “catalytic activity” herein is meant a chemical reaction that can be catalyzed by an enzyme. The chemical reaction may be one that already exists in nature, i.e., a known reaction such as hydrolysis, or the chemical reaction may not exist in nature, i.e., an unknown reaction such as a chemical reaction designed to make or degrade a synthetic compound such as polyester, the use of histidine to catalyze ester hydrolysis (see Examples). By “ligand binding activity” herein is meant a domain that can bind a ligand, but may be catalytically inert toward that ligand. That is the domain has the functional groups to bind a ligand, but lacks the functional groups to engage in catalysis. By “enzyme” herein is meant proteins that bring one or more substrates together in an optimal orientation as a prelude to the making and breaking of chemical bonds. Thus, the active site of an enzyme is the region (also referred to herein as domain) that binds the substrates and contains the residues that directly participate in the making and breaking of chemical bonds.

Thus, a number of active site domains can be designed using the computational methods described herein. For example, active site domains that mimic naturally occurring (i.e. known) chemical reactions, such as bond breaking, acyl-group transfers, phosphoryl-group transfers, and glycosyl transfers, modeled on known enzymatic principles can be designed and inserted into a protein scaffold to generate a protein with enzymes-like activity. Active site domains may be obtained from any number of enzymes. Suitable classes of enzymes from which active site domains may be obtained include, but are not limited to, hydrolases such as proteases, carbohydrases, lipases and nucleases; isomerases such as racemases, epimerases, tautomerases, or mutases; transferases, kinases and phophatases. Preferably, the design process results in enzymes that have enhanced catalytic rates of reaction.

Likewise, de novo active site domains may be designed and inserted into a protein scaffold to generate proteins with novel enzyme-like activities. For example, de novo active site domains may catalyze a known reaction (e.g., hydrolysis) using different catalytic residues or functional groups. Alternatively, de novo active site domains may catalyze a reaction not known in nature. That is, an active site domain may be constructed based on purely physical principles to transform either a naturally occurring or synthetic substrate. In some embodiments, the substrate is one that has not previously been susceptible to enzymatic catalysis.

Finally, active site domains may be designed and inserted into a protein scaffold to generate ligand binding proteins. Ligand binding proteins contain active site domains that lack catalytic activity, but bind to a substrate or an inhibitor. Ligand binding proteins may be designed using the same computational methods described herein except that the rotamers used to build the active site domain are ground state (or low energy state) rather than high energy state rotamers.

As will be appreciated by those skilled in the art, the potential list of suitable enzyme active site domains is quite large. Thus, active site domains may obtained from enzymes such as lactase, maltase, sucrase or invertase, cellulase, α-amylase, aldolases, glycogen phosphorylase, kinases such as hexokinase, proteases such as serine, cysteine, aspartyl and metalloproteases, including, but not limited to, trypsin, chymotrypsin, and other therapeutically relevant serine proteases such as tPA; cysteine proteases including the cathepsins, e.g., cathepsin B, L, S, H, J, N and O; calpain; and caspases, e.g., caspase-3, -5, -8 and other caspases of the apoptotic pathway, and, interleukin-converting enzyme (ICE). Particularly preferred are active site domains from enzymes used as indicators of or treatment for: (1) heart disease, including creatine kinase, lactate dehydrogenase, aspartate amino transferase, troponin T, myoglobin, fibrinogen, cholesterol, triglycerides, thrombin, tissue plasminogen activator (tPA); (2) pancreatic disease indicators including amylase, lipase, chymotrypsin and trypsin; (3) liver function enzymes and proteins including cholinesterase, bilirubin, and alkaline phosphotase; aldolase, prostatic acid phosphatase, terminal deoxynucleotidyl transferase, and (4) bacterial and viral enzymes such as HIV protease. As will be appreciated in the art, this list is not meant to be limiting.

In a preferred embodiment, the active site domain is a ligand binding domain. As will be appreciated by those in the art, there are a wide variety of ligand/binding partners known in the art. In this embodiment, the ligand may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc.

These ligand binding protozymes find use in a variety of applications, including in biosensors for the detection of the ligand (e.g. biosensors for the detection of toxic ligands or spores, or biosensors for the diagnosis, e.g. the presence or absence of therapeutic molecules in patient samples); or in therapeutic applications, such as the “absorption” of therapeutically undesirable molecules by competing with the natural binding partner for the natural ligand.

Once a scaffold protein and an active site domain is chosen, a set of candidate variant proteins with putative enzyme-like activity is generated. By “candidate variant protein” herein is meant enzyme-like proteins, i.e., protozymes that have been designed using the computational methods outlined herein to differ from the corresponding scaffold protein by at least 1 amino acid. Preferably, the candidate variant protein sequences are generally different from the scaffold sequence in regions critical for catalytic activity. Preferably, the candidate variant protein exhibits a known catalytic activity, that may be the same or different from the wild-type enzyme. More preferably, the candidate variant protein exhibits a new catalytic activity using different catalytic residues to catalyze a known reaction or different catalytic residues to catalyze an unknown reaction. More preferably, the candidate variant protein exhibits ligand binding activity.

Alternatively, primary libraries, e.g., libraries of all or a subset of possible candidate variant protein sequence with putative catalytic activity is generated. In a preferred embodiment, some subset of the primary library is then experimentally generated to form a secondary library. Alternatively, some or all of the primary library members are recombined to form a secondary library, e.g., with new members. Again, this may be done either computationally or experimentally or both.

As will be appreciated by those of skill in the art, candidate variant proteins with putative catalytic activity may be generated by selecting an appropriate scaffold, choosing an active site domain and then using the computational methods described below to insert the active site domain and to change the identity of the surrounding amino acids to other amino acids to optimize the catalytic reaction.

Alternatively, variant proteins with putative catalytic activity may be generated by choosing an active site domain and using structural homology methods to pick an appropriate scaffold. Once an appropriate scaffold is selected, computational methods can be used to insert the active site domain and change the identity of the surrounding amino acids to other amino acids to optimize the catalytic reaction.

Generally, there are a variety of computational protein design cycle methods that can be used to generate a set of candidate variant proteins with putative catalytic activity. By “protein design cycle” herein is meant any one of a number of protein design algorithms that can be used to produce a sequence or sequences including but not limited to sequence based methods and structural based methods such as Protein Design Automation (PDA™), described in detail below, are used. Other methods for assessing the relative energies of sequences with high precision include Warshel, computer Modeling of Chemical Reactions in Enzymes and Solutions, Wiley & Sons, New York, (1991), hereby expressly incorporated by reference.

Sequence based alignments can be used in a variety of ways. For example, a numberof related proteins can be aligned, as is known in the art, and the “variable” and “conserved” residues defined; that is, the residues that vary or remain identical between the family members can be defined. These results can be used to generate a probability table, as outlined below. Similarly, these sequence variations can be tabulated and a secondary library defined from them as defined below. Alternatively, the allowed sequence variations can be used to define the amino acids considered at each position during the computational screening. Another variation is to bias the score for amino acids that occur in the sequence alignment, thereby increasing the likelihood that they are found during computational screening but still allowing consideration of other amino acids. This bias would result in a focused primary library but would not eliminate from consideration amino acids not found in the alignment. In addition, a number of other types of bias may be introduced. For example, diversity may be forced; that is, a “conserved” residue is chosen and altered to force diversity on the protein and thus sample a greater portion of the sequence space. Alternatively, the positions of high variability between family members (i.e. low conservation) can be randomized, either using all or a subset of amino acids. Similarly, outlier residues, either positional outliers or side chain outliers, may be eliminated.

Similarly, structural alignment of structurally related proteins can be done to generate sequence alignments. There are a wide variety of such structural alignment programs known. See for example VAST from the NCBI (http://www.ncbi.nlm.nih.gov:80/Structure/VAST/vast.shtml); SSAP (Orengo and Taylor, Methods Enzymol 266(617-635 (1996)) SARF2 (Alexandrov, Protein Eng 9(9):727-732. (1996)) CE (Shindyalov and Bourne, Protein Eng 11(9):739-747. (1998)); (Orengo et al., Structure 5(8):1093-108 (1997); Dali (Holm et al., Nucleic Acid Res. 26(1):316-9 (1998), all of which are incorporated by reference). These structurally-generated sequence alignments can then be examined to determine the observed sequence variations.

Libraries of primary variant sequences can be generated by predicting secondary structure from sequence, and then selecting sequences that are compatible with the predicted secondary structure. There are a number of secondary structure prediction methods, including, but not limited to, threading (Bryant and Altschul, Curr Opin Struct Biol 5(2):236-244. (1995)), Profile 3D (Bowie, et al., Methods Enzymol 266(598-616 (1996); MONSSTER (Skolnick, et al., J Mol Biol 265(2):217-241. (1997); Rosetta (Simons, et al., Proteins 37(S3):171-176 (1999); PSI-BLAST (Altschul and Koonin, Trends Biochem Sci 23(11):444-447. (1998)); Impala (Schaffer, et al., Bioinformatics 15(12):1000-1011. (1999)); HMMER (McClure, et al., Proc Int Conf Intell Syst Mol Biol 4(155-164 (1996)); Clustal W (http://www.ebi.ac.uk/clustalw/); BLAST (Altschul, et al., J Mol Biol 215(3):403-410. (1990)), helix-coil transition theory (Munoz and Serrano, Biopolymers 41:495, 1997), neural networks, local structure alignment and others (e.g., see in Selbig et al., Bioinformatics 15:1039, 1999).

Similarly, as outlined above, other computational methods are known, including, but not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994); PROSA (Heindlich et al., J. Mol. Biol. 216:167-180 (1990); THREADER (Jones et al., Nature 358:86-89 (1992), and other inverse folding methods such as those described by Simons et al. (Proteins, 34:535-543, 1999), Levitt and Gerstein (PNAS USA, 95:5913-5920, 1998), Godzik et al., PNAS, V89, PP 12098-102; Godzik and Skolnick (PNAS USA, 89:12098-102, 1992), Godzik et al. (J. Mol. Biol. 227:227-38, 1992) and two profile methods (Gribskov et al. PNAS 84:4355-4358 (1987) and Fischer and Eisenberg, Protein Sci. 5:947-955 (1996), Rice and Eisenberg J. Mol. Biol. 267:1026-1038(1997)), all of which are expressly incorporated by reference. In addition, other computational methods such as those described by Koehl and Levitt (J. Mol. Biol. 293:1161-1181 (1999); J. Mol. Biol. 293:1183-1193 (1999); expressly incorporated by reference) can be used to create a protein sequence library which can optionally then be used to generate a smaller secondary library for use in experimental screening for improved properties and function.

In addition, there are computational methods based on force field calculations such as SCMF that can be used as well. For SCMF, see Delarue et la. Pac. Symp. Biocomput. 109-21 (1997), Koehl et al., J. Mol. Biol. 239:249 (1994); Koehl et al., Nat. Struc. Biol. 2:163 (1995); Koehl et al., Curr. Opin. Struct. Biol. 6:222 (1996); Koehl et al., J. Mol. Bio. 293:1183 (1999); Koehl et al., J. Mol. Biol. 293:1161 (1999); Lee J. Mol. Biol. 236:918 (1994); and Vasquez Biopolymers 36:53-70 (1995); all of which are expressly incorporated by reference. Other force field calculations that can be used to optimize the conformation of a sequence within a computational method, or to generate de novo optimized sequences as outlined herein include, but are not limited to, OPLS-AA (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem 1994 May;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al., (1988) Proteins: Structure, Function and Genetics, v4, pp31-47); cff91 (Maple, et al., J. Comp. Chem. v15, 162-182); also, the DISCOVER (cvff and cff91) and AMBER force fields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference. In fact, as is outlined below, these force field methods may be used to generate the secondary library directly; that is, no primary library is generated; rather, these methods can be used to generate a probability table from which the secondary library is directly generated, for example by using these forcefields during an SCMF calculation.

In a preferred embodiment, the computational method used to generate the primary library is Protein Design Automation™ (PDA™) technology, as is described in U.S. Ser. Nos. 60/061,097, 60/043,464, 60/054,678, 09/127,926, 09/782,004 and PCT US98/07254, all of which are expressly incorporated herein by reference. Other names for PDA™ include ORBIT (Optimization of Rotamers By Iterative Techniques; see Dahiyat, B. I., & Mayo, S. L., (1997) Science, 278:82-87).

Briefly, the PDA™ protein design technology can be described as follows: A known protein structure for the position (for example, core residues generally will be selected from the set of hydrophobic residues, surface residues generally will be selected from the hydrophilic residues, and boundary residues may be either). Each amino acid can be represented by a discrete set of all allowed conformers of each side-chain, called rotamers. Thus, to arrive at an optimal sequence for a backbone, all possible sequences of rotamers must be screened, where each backbone position can be occupied either by each amino acid in all its possible rotameric states, or a subset of amino acids, and thus a subset of rotamers.

In addition to rotamers describing amino acid side chain conformations, the computational method described herein requires a set of rotamers be generated that represent the desired catalytic (or ligand binding) function. For designs directed at generating enzyme-like proteins, a set of rotamers representing some high energy state of the substrate is generated. The high energy state of the substrate can include the transition state of a targeted chemical reaction, or some intermediate (high or low energy) state on the reaction pathway of a targeted chemical reaction. For designs directed at generating ligand binding proteins, a set of rotamers representing some low energy or ground state is generated.

The high energy state is a state similar to the transition state for the targeted chemical reaction. “High energy state” is meant to include high energy states of the substrate on some reaction pathway, high energy states of the substrate/protein complex on some reaction pathway, transition states of the substrate on some reaction pathway, transition states of the substrate/protein complex on some reaction pathway, intermediate states of the substrate on some reaction pathway, intermediate states of the substrate/protein complex on some reaction pathway, low energy states of the substrate, low energy states of the substrate/protein complex, ground states of the substrate, and ground states of the substrate/protein complex.

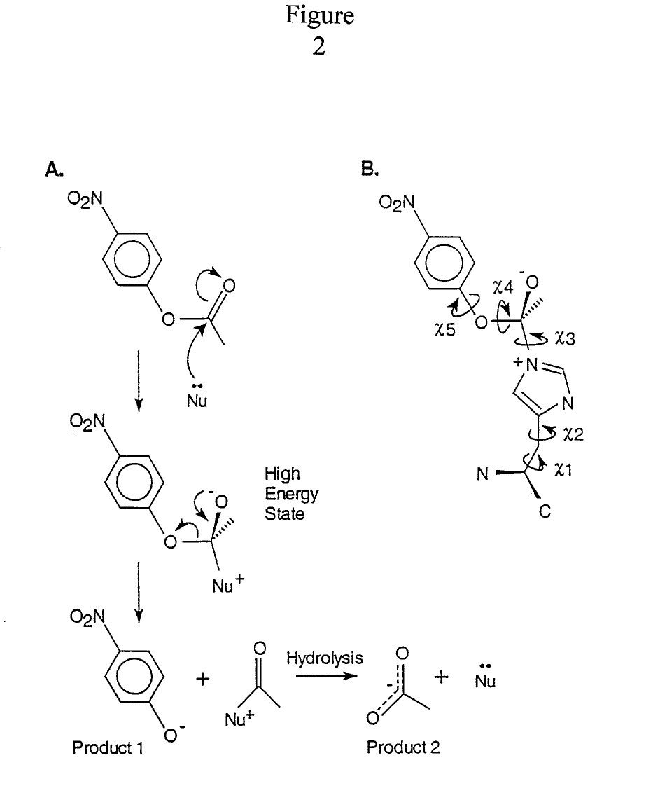

In a preferred embodiment, the high energy state rotamers are generated in manner that directly includes interactions with certain amino acid side chains, for example, see FIG. 2B. As will be appreciated by those skilled in the art, direct attachment of the substrate in a high energy state configuration to an amino acid has the benefit of restricting the resulting search space to those substrate/amino acid orientations that are likely to occur on the reaction pathway or in ligand binding.

In addition to rotations required to describe the amino acid side chain conformations, rotations about dihedral angles internal to the substrate and rotations about dihedral angles resulting from attaching the substrate to the amino acid are included. In a preferred embodiment, the values for these dihedral angle rotations are obtained from an analysis of the structures of known compounds. In an alternative embodiment, the values for these dihedral angle rotations are obtained from a consideration of the chemical nature of the high energy state.

As will be appreciated by those skilled in the art, the high energy state rotamers need not be directly attached to an amino acid. The high energy state rotamers can be generated using a three dimensional grid of points that span the catalytic domain. In a preferred embodiment, the lattice spacing for this grid of points is 0.5 angstroms. Or more preferably, the lattice spacing is 0.1 angstroms. Or even more preferably, the lattice spacing is set at a value that results in a calculation whose combinatorial complexity is tractable. For each grid point, the substrate is subjected to rotations in the X, Y, and Z dimensions. In a preferred embodiment, the X, Y, and Z rotations are done in rotation increments of 30 degrees. Or more preferably, the X, Y, and Z rotations are done in rotation increments of 5 degrees. Or even more preferably, the X, Y, and Z rotations are done using rotation increments that result in a calculation whose combinatorial complexity is tractable. The X, Y, and Z rotations can be nested or un-nested. In addition, the dihedral angles internal to the substrate can be varied.

In a preferred embodiment, the size of the grid (including consideration for lattice spacing), the X, Y, and Z rotation increments (including consideration for nested rotations), and the internal dihedral angle values for the substrate are selected so that the combinatorial complexity of the resulting calculation is tractable. As will be appreciated by those skilled in the art, calculations that involve a combination of high energy state rotamers generated by direct attachment of the substrate to an amino acid (or amino acids) and high energy state rotamers generated by the grid based method described above are possible.

Two sets of interactions are then calculated for each rotamer at every position: the interaction of the rotamer side chain with all or part of the backbone (the “singles” energy, also called the rotamer/template or rotamer/backbone energy), and the interaction of the rotamer side chain with all other possible rotamers at every other position or a subset of the other positions (the “doubles” energy, also called the rotamer/rotamer energy). The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a “Dead End Elimination” (DEE) calculation is performed. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence that represents the global optimum energy.

Once the global solution has been found, a Monte Carlo search may be done to generate a rank-ordered list of sequences in the neighborhood of the DEE solution. Starting at the DEE solution, random positions are changed to other rotamers, and the new sequence energy is calculated. If the new sequence meets the criteria for acceptance, it is used as a starting point for another jump. After a predetermined number of jumps, a rank-ordered list of sequences is generated.

Monte Carlo searching is a sampling technique to explore sequence space around the global minimum or to find new local minima distant in sequence space. As is more additionally outlined below, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Similarly, for all the sampling techniques, the acceptance criteria of whether a sampling jump is accepted can be altered.

As outlined in U.S. Ser. No. 09/127,926, the protein backbone (comprising (for a naturally occurring protein) the nitrogen, the carbonyl carbon, the α-carbon, and the carbonyl oxygen, along with the direction of the vector from the α-carbon to the β-carbon) may be altered prior to the computational analysis, by varying a set of parameters called supersecondary structure parameters.

Once a protein structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths. In a preferred embodiment, this is done by doing a number of steps of conjugate gradient minimization (Mayo et al., J. Phys. Chem. 94:8897 (1990)) of atomic coordinate positions to minimize the Dreiding force field with no electrostatics. Generally from about 10 to about 250 steps is preferred, with about 50 being most preferred.

The protein backbone structure contains at least one variable residue position. As is known in the art, the residues, or amino acids, of proteins are generally sequentially numbered starting with the N-terminus of the protein. Thus a protein having a methionine at it's N-terminus is said to have a methionine at residue or amino acid position 1, with the next residues as 2, 3, 4, etc. At each position, the wild type (i.e. naturally occurring) protein may have one of at least 20 amino acids, in any number of rotamers. By “variable residue position” herein is meant an amino acid position of the protein to be designed that is not fixed in the design method as a specific residue or rotamer, generally the wild-type residue or rotamer.

In a preferred embodiment, all of the residue positions of the protein are variable. That is, every amino acid side chain may be altered in the methods of the present invention. This is particularly desirable for smaller proteins, although the present methods allow the design of larger proteins as well. While there is no theoretical limit to the length of the protein that may be designed this way, there is a practical computational limit.

In an alternate preferred embodiment, only some of the residue positions of the protein are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable (for example, to eliminate a proteolytic site or alter the substrate specificity of an enzyme), the residue may be fixed as a particular amino acid.

Alternatively, the methods of the present invention may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In a preferred embodiment, residues that can be fixed include, but are not limited to, structurally or biologically functional residues; alternatively, biologically function residues may specifically not be fixed. For example, residues which are known to be important for biological activity, such as the residues which form the active site of an enzyme, the substrate binding site of an enzyme, the binding site for a binding partner (ligand/receptor, antigen/antibody, etc.), phosphorylation or glycosylation sites which are crucial to biological function, or structurally important residues, such as disulfide bridges, metal binding sites, critical hydrogen bonding residues, residues critical for backbone conformation such as proline or glycine, residues critical for packing interactions, etc. may all be fixed in a conformation or as a single rotamer, or “floated”.

Similarly, residues which may be chosen as variable residues may be those that confer undesirable biological attributes, such as susceptibility to proteolytic degradation, dimerization or aggregation sites, glycosylation sites which may lead to immune responses, unwanted binding activity, unwanted allostery, undesirable enzyme activity but with a preservation of binding, etc.

In a preferred embodiment, each variable position is classified as either a core, surface or boundary residue position, although in some cases, as explained below, the variable position may be set to glycine to minimize backbone strain. In addition, as outlined herein, residues need not be classified, they can be chosen as variable and any set of amino acids may be used. Any combination of core, surface and boundary positions can be utilized: core, surface and boundary residues; core and surface residues; core and boundary residues, and surface and boundary residues, as well as core residues alone, surface residues alone, or boundary residues alone.

The classification of residue positions as core, surface or boundary may be done in several ways, as will be appreciated by those in the art. In a preferred embodiment, the classification is done via a visual scan of the original protein backbone structure, including the side chains, and assigning a classification based on a subjective evaluation of one skilled in the art of protein modeling. Alternatively, a preferred embodiment utilizes an assessment of the orientation of the Cα-Cβ vectors relative to a solvent accessible surface computed using only the template Ca atoms, as outlined in U.S. Ser. Nos. 60/061,097, 60/043,464, 60/054,678, 09/127,926 and PCT US98/07254. Alternatively, a surface area calculation can be done.

Once each variable position is classified as core, surface or boundary, a set of amino acid side chains, and thus a set of rotamers, is assigned to each position. That is, the set of possible amino acid side chains that the program will allow to be considered at any particular position is chosen. Subsequently, once the possible amino acid side chains are chosen, the set of rotamers that will be evaluated at a particular position can be determined. Thus, a core residue will generally be selected from the group of hydrophobic residues consisting of alanine, valine, isoleucine, leucine, phenylalanine, tyrosine, tryptophan, and methionine (in some embodiments, when the α scaling factor of the van der Waals scoring function, described below, is low, methionine is removed from the set), and the rotamer set for each core position potentially includes rotamers for these eight amino acid side chains (all the rotamers if a backbone independent library is used, and subsets if a rotamer dependent backbone is used). Similarly, surface positions are generally selected from the group of hydrophilic residues consisting of alanine, serine, threonine, aspartic acid, asparagine, glutamine, glutamic acid, arginine, lysine and histidine. The rotamer set for each surface position thus includes rotamers for these ten residues. Finally, boundary positions are generally chosen from alanine, serine, threonine, aspartic acid, asparagine, glutamine, glutamic acid, arginine, lysine histidine, valine, isoleucine, leucine, phenylalanine, tyrosine, tryptophan, and methionine. The rotamer set for each boundary position thus potentially includes every rotamer for these seventeen residues (assuming cysteine, glycine and proline are not used, although they can be). Additionally, in some preferred embodiments, a set of 18 naturally occurring amino acids (all except cysteine and proline, which are known to be particularly disruptive) are used.

Thus, as will be appreciated by those in the art, there is a computational benefit to classifying the residue positions, as it decreases the number of calculations. It should also be noted that there may be situations where the sets of core, boundary and surface residues are altered from those described above; for example, under some circumstances, one or more amino acids is either added or subtracted from the set of allowed amino acids. For example, some proteins that dimerize or multimerize, or have ligand-binding sites, may contain hydrophobic surface residues, etc. In addition, residues that do not allow helix “capping” or the favorable interaction with an α-helix dipole may be subtracted from a set of allowed residues. This modification of amino acid groups is done on a residue by residue basis.

In a preferred embodiment, proline, cysteine and glycine are not included in the list of possible amino acid side chains, and thus the rotamers for these side chains are not used. However, in a preferred embodiment, when the variable residue position has a φ angle (that is, the dihedral angle defined by 1) the carbonyl carbon of the preceding amino acid; 2) the nitrogen atom of the current residue; 3) the α-carbon of the current residue; and 4) the carbonyl carbon of the current residue) greater than 0°, the position is set to glycine to minimize backbone strain.

Once the group of potential rotamers is assigned for each variable residue position, processing proceeds as outlined in U.S. Ser. No. 09/127,926 and PCT US98/07254. This processing step entails analyzing interactions of the rotamers with each other and with the protein backbone to generate optimized protein sequences. Simplistically, the processing initially comprises the use of a number of although the scoring functions may differ depending on the position classification or other considerations, like favorable interaction with an α-helix dipole. As outlined below, the total energy which is used in the calculations is the sum of the energy of each scoring function used at a particular position, as is generally shown in Equation 1:

Eₜₒₜₐₗ=nE_{vdw}+nEₐₛ+nE_{h-bonding}+nEₛₛ+nE_{elec} Equation 1

In Equation 1, the total energy is the sum of the energy of the van der Waals potential (E_{vdw}), the energy of atomic solvation (Eₐₛ), the energy of hydrogen bonding (E_{h-bonding}), the energy of secondary structure (Eₛₛ) and the energy of electrostatic interaction (E_{elec}). The term n is either 0 or 1, depending on whether the term is to be considered for the particular residue position. Alternatively, n can be a non integral value.

As outlined in U.S. Ser. Nos. 60/061,097, 60/043,464, 60/054,678, 09/127,926 and PCT US98/07254, any combination of these scoring functions, either alone or in combination, may be used. Once the scoring functions to be used are identified for each variable position, the preferred first step in the computational analysis comprises the determination of the interaction of each possible rotamer with all or part of the remainder of the protein. That is, the energy of interaction, as measured by one or more of the scoring functions, of each possible rotamer at each variable residue position with either the backbone or other rotamers, is calculated. In a preferred embodiment, the interaction of each rotamer with the entire remainder of the protein, i.e. both the entire template and all other rotamers, is done. However, as outlined above, it is possible to only model a portion of a protein, for example a domain of a larger protein, and thus in some cases, not all of the protein need be considered. The term “portion”, as used herein, with regard to a protein refers to a fragment of that protein. This fragment may range in size from 10 amino acid residues to the entire amino acid sequence minus one amino acid. Accordingly, the term “portion”, as used herein, with regard to a nucleic refers to a fragment of that nucleic acid. This fragment may range in size from 10 nucleotides to the entire nucleic acid sequence minus one nucleotide.

In a preferred embodiment, the first step of the computational processing is done by calculating two sets of interactions for each rotamer at every position: the interaction of the rotamer side chain with the template or backbone (the “singles” energy), and the interaction of the rotamer side chain with all other possible rotamers at every other position (the “doubles” energy), whether that position is varied or floated. It should be understood that the backbone in this case includes both the atoms of the protein structure backbone, as well as the atoms of any fixed residues, wherein the fixed residues are defined as a particular conformation of an amino acid.

Thus, “singles” (rotamer/template) energies are calculated for the interaction of every possible rotamer at every variable residue position with the backbone, using some or all of the scoring functions. Thus, for the hydrogen bonding scoring function, every hydrogen bonding atom of the rotamer and every hydrogen bonding atom of the backbone is evaluated, and the E_{HB} is calculated for each possible rotamer at every variable position. Similarly, for the van der Waals scoring function, every atom of the rotamer is compared to every atom of the template (generally excluding the backbone atoms of its own residue), and the E_{vdW} is calculated for each possible rotamer at every variable residue position. In addition, generally no van der Waals energy is calculated if the atoms are connected by three bonds or less. For the atomic solvation scoring function, the surface of the rotamer is measured against the surface of the template, and the Eₐₛ for each possible rotamer at every variable residue position is calculated. The secondary structure propensity scoring function is also considered as a singles energy, and thus the total singles energy may contain an Eₛₛ term. As will be appreciated by those in the art, many of these energy terms will be close to zero, depending on the physical distance between the rotamer and the template position; that is, the farther apart the two moieties, the lower the energy.

For the calculation of “doubles” energy (rotamer/rotamer), the interaction energy of each possible rotamer is compared with every possible rotamer at all other variable residue positions. Thus, “doubles” energies are calculated for the interaction of every possible rotamer at every variable residue position with every possible rotamer at every other variable residue position, using some or all of the scoring functions. Thus, for the hydrogen bonding scoring function, every hydrogen bonding atom of the first rotamer and every hydrogen bonding atom of every possible second rotamer is evaluated, and the E_{HB} is calculated for each possible rotamer pair for any two variable positions. Similarly, for the van der Waals scoring function, every atom of the first rotamer is compared to every atom of every possible second rotamer, and the E_{vdW} is calculated for each possible rotamer pair at every two variable residue positions. For the atomic solvation scoring function, the surface of the first rotamer is measured against the surface of every possible second rotamer, and the Eₐₛ for each possible rotamer pair at every two variable residue positions is calculated. The secondary structure propensity scoring function need not be run as a “doubles” energy, as it is considered as a component of the “singles” energy. As will be appreciated by those in the art, many of these double energy terms will be close to zero, depending on the physical distance between the first rotamer and the second rotamer; that is, the farther apart the two moieties, the lower the energy.

Computational design algorithms that also may be used to generate candidate variant proteins with novel catalytic functions include the sequence prediction algorithm (SPA) as described in Raha, K., et al. (2000) Protein Sci., 9:1106-1119, expressly incorporated herein by reference.

In addition, as will be appreciated by those in the art, a variety, of force fields can be used in the PDA™ technology calculations, including, but not limited to, Dreiding I and Dreiding II (Mayo et al, J. Phys. Chem. 948897 (1990)), AMBER (Weiner et al., J. Amer. Chem. Soc. 106:765 (1984) and Weiner et al., J. Comp. Chem. 106:230 (1986)), MM2 (Allinger J. Chem. Soc. 99:8127 (1977), Liljefors et al., J. Com. Chem. 8:1051 (1987)); MMP2 (Sprague et al., J. Comp. Chem. 8:581 (1987)); CHARMM (Brooks et al., J. Comp. Chem. 106:187 (1983)); GROMOS; and MM3 (Allinger et al., J. Amer. Chem. Soc. 111:8551 (1989)), OPLS-AA (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPP/3 (Liwo et al., J Protein Chem 1994 May;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al.,(1988) Proteins: Structure, Function and Genetics, v4, pp3147); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.), all of which are expressly incorporated by reference.

Once the singles and doubles energies are calculated and stored, the next step of the computational processing may occur. As outlined in U.S. Ser. No. 09/127,926 and PCT US98/07254, preferred embodiments utilize a Dead End Elimination (DEE) step, and preferably a Monte Carlo step. PDA™ technology, viewed broadly, has three components that may be varied to alter the output (e.g. the primary library): the scoring functions used in the process; the filtering technique, and the sampling technique. These functions may be used sequentially or substantially simultaneously. For example, a scoring function may be used in parallel with a filtering technique.

In a preferred embodiment, the scoring functions may be altered. In a preferred embodiment, the scoring functions outlined above may be biased or weighted in a variety of ways. For example, a bias towards or away from a reference sequence or family of sequences can be done; for example, a bias towards wild-type or homolog residues may be used. Similarly, the entire protein or a fragment of it may be biased; for example, the active site may be biased towards wild-type residues, or domain residues towards a particular desired physical property can be done. Furthermore, a bias towards or against increased energy can be generated. Additional scoring function biases include, but are not limited to applying electrostatic potential gradients or hydrophobicity gradients, adding a substrate or binding partner to the calculation, or biasing towards a desired charge or hydrophobicity.

In addition, in an alternative embodiment, there are a variety of additional scoring functions that may be used. Additional scoring functions include, but are not limited to torsional potentials, or residue pair potentials, or residue entropy potentials. Such additional scoring functions can be used alone, or as functions for processing the library after it is scored initially.

In a preferred embodiment, a variety of process filtering techniques can be done, including, but not limited to, DEE and its related counterparts. Additional filtering techniques include, but are not limited to branch-and-bound techniques for finding optimal sequences (Gordon and Mayo, Structure Fold. Des. 7:1089-98, 1999), and exhaustive enumeration of sequences. It should be noted however, that some techniques may also be done without any filtering techniques; for example, sampling techniques can be used to find good sequences, in the absence of filtering.

As will be appreciated by those in the art, once an optimized sequence or set of sequences is generated, (or again, these need not be optimized or ordered) a variety of sequence space sampling methods can be done, either in addition to the preferred Monte Carlo methods, or instead of a Monte Carlo search. That is, once a sequence or set of sequences is generated, preferred methods utilize sampling techniques to allow the generation of additional, related sequences for testing.

These sampling methods can include the use of amino acid substitutions, insertions or deletions, or recombinations of one or more sequences. As outlined herein, a preferred embodiment utilizes a Monte Carlo search, which is a series of biased, systematic, or random jumps. However, there are other sampling techniques that can be used, including Boltzman sampling, genetic algorithm techniques and simulated annealing. In addition, for all the sampling techniques, the kinds of jumps allowed can be altered (e.g. random jumps to random residues, biased jumps (to or away from wild-type, for example), jumps to biased residues (to or away from similar residues, for example), etc.). Jumps where multiple residue positions are coupled (two residues always change together, or never change together), jumps where whole sets of residues change to other sequences (e.g., recombination). Similarly, for all the sampling techniques, the acceptance criteria of whether a sampling jump is accepted can be altered, to allow broad searches at high temperature and narrow searches close to local optima at low temperatures. See Metropolis et al., J. Chem Phys v21, pp 1087, 1953, hereby expressly incorporated by reference.

In addition, it should be noted that the preferred methods of the invention result in a rank ordered list of sequences; that is, the sequences are ranked or filtered on the basis of some objective criteria. However, as outlined herein, it is possible to create a set of non-ordered sequences, for example by generating a probability table directly (for example using SCMF analysis or sequence alignment techniques) that lists sequences without ranking them. The sampling techniques outlined herein can be used in either situation.

In a preferred embodiment, Boltzman sampling is done. As will be appreciated by those in the art, the temperature criteria for Boltzman sampling can be altered to allow broad searches at high temperature and narrow searches close to local optima at low temperatures (see e.g., Metropolis et al., J. Chem. Phys. 21:1087, 1953).

In a preferred embodiment, the sampling technique utilizes genetic algorithms, e.g., such as those described by Holland (Adaptation in Natural and Artificial Systems, 1975, Ann Arbor, U. Michigan Press). Genetic algorithm analysis generally takes generated sequences and recombines them computationally, similar to a nucleic acid recombination event, in a manner similar to “gene shuffling”. Thus the “jumps” of genetic algorithm analysis generally are multiple position jumps. In addition, as outlined below, correlated multiple jumps may also be done. Such jumps can occur with different crossover positions and more than one recombination at a time, and can involve recombination of two or more sequences. Furthermore, deletions or insertions (random or biased) can be done. In addition, as outlined below, genetic algorithm analysis may also be used after the secondary library has been generated.

In a preferred embodiment, the sampling technique utilizes simulated annealing, e.g., such as described by Kirkpatrick et al. (Science, 220:671-680, 1983). Simulated annealing alters the cutoff for accepting good or bad jumps by altering the temperature. That is, the stringency of the cutoff is altered by altering the temperature. This allows broad searches at high temperature to new areas of sequence space, altering with narrow searches at low temperature to explore regions in detail.

In addition, as outlined below, these sampling methods can be used to further process a secondary library to generate additional secondary libraries (sometimes referred to herein as tertiary libraries).

As will be appreciated by those of skill in the art, any protein design cycle can be used individually, in combination with other methods, or in reiterations that combine methods.

Thus, sets of candidate variant proteins or primary libraries comprising all or a subset of candidate variant proteins can be generated in variety of computational ways (i.e., using a variety of protein design cycles), including structure based methods such as PDA™, or sequence based methods, or combinations as outlined herein.

In a preferred embodiment, sets of candidate variant proteins or primary libraries are generated using PDA™. As will be appreciated by those of skill in the art, inserting the active domain and analyzing the surrounding amino acids for optimization of the active site domain may be done in any order or at the same time.

In a preferred embodiment, the computational processing results in a set of optimized variant candidate sequences with putative enzyme-like activity. The optimized variant candidate protein sequences are generally different from the scaffold protein sequence in regions critical to enzymatic activity, i.e., the active site domain. Preferably, each optimized variant candidate sequence comprises at least one variant amino acid from the scaffold, with 3 to 5 being preferred.

Accordingly, in a preferred embodiment, the present invention is directed to methods of computationally processing a scaffold protein, or fragment thereof, to produce a variant candidate protein, a set of variant candidate protein sequences, or a primary library of variant protein sequences.

In a preferred embodiment, the variant candidate proteins of the invention have an amino acid sequence that differs from the scaffold protein due to the incorporation of one or more catalytic residues. Preferably, the variant candidate proteins also differ from the scaffold protein due to the presence of amino acids necessary for substrate recognition and binding

Accordingly, the computational processing results in a set of primary variant sequences, that may be optimized protein sequences if some sort of ranking or scoring functions are used. These optimized protein sequences are generally, but not always, significantly different from the scaffold sequence from which the backbone was taken. That is, each optimized protein sequence preferably comprises at least about 5-10% variant amino acids from the starting scaffold or wild-type scaffold, with at least about 15-20% changes being preferred and at least about 30% changes being particularly preferred.

In a preferred embodiment, at least one candidate variant protein is identified with putative enzyme-like activity. Any method of identifying potential or actual enzymatic activity can be used in the invention. Acceptable methods include computational or physical methods. For example, computational methods can be used to identify catalytic sites within a protein structure as well as the residues necessary to accommodate substrate binding (Bolon and Mayo, (2001) Proc Natl Acad Sci USA, 98:14274-14279; incorporated herein by reference). Acceptable experimental methods include determination of “burst” phase kinetics at high substrate concentrations, and determination of kinetic parameters, such as the K_{M} (Bolon and Mayo, (2001) Proc Natl Acad Sci USA, 98: 14274-14279).

Having identified potential enzyme-like sequences, these sequences can then be modified by the replacement of one or more amino acids as described below. Once the candidate variant protein has been so modified, the protein is then tested to determine if its activity is similar to the wild type enzyme from which the active site domain was obtained (see, for example Bolon and Mayo, (2001) Proc Natl Acad Sci USA, 98: 14274-14279). The variant may retain full activity, or retain a sufficient proportion of its activity to be useful.

The variant proteins and nucleic acids of the invention are distinguishable from the naturally occurring target protein. By “naturally occurring” or “wild type” or grammatical equivalents, herein is meant an amino acid sequence or a nucleotide sequence that is found in nature and includes allelic variations; that is, an amino acid sequence or a nucleotide sequence that usually has not been intentionally modified. Accordingly, by “non-naturally occurring” or “synthetic” or “recombinant” or grammatical equivalents thereof, herein is meant an amino acid sequence or a nucleotide sequence that is not found in nature; that is, an amino acid sequence or a nucleotide sequence that usually has been intentionally modified. It is understood that once a recombinant nucleic acid is made and reintroduced into a host cell or organism, it will replicate non-recombinantly, i.e., using the in vivo cellular machinery of the host cell rather than in vitro manipulations, however, such nucleic acids, once produced recombinantly, although subsequently replicated non-recombinantly, are still considered recombinant for the purpose of the invention. Thus, the variant proteins and nucleic acids of the invention are non-naturally occurring; that is, they do not exist in nature.

Thus, in a preferred embodiment, the variant protein has an amino acid sequence that differs from a target sequence by at least 1-5% of the residues. That is, the variant proteins of the invention are less than about 97-99% identical to a target amino acid sequence. Accordingly, a protein is a “candidate variant protein” if the overall homology of the protein sequence to the target sequence is preferably less than about 99%, more preferably less than about 98%, even more preferably less than about 97% and more preferably less than about 95%. In some embodiments, the homology will be as low as about 75-80%.

Homology in this context means sequence similarity or identity, with identity being preferred. As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc. All references cited in this paragraph are incorporated by reference in their entirety.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989). Useful PILEUP parameters including a default gap weight of 3.00, a default gap length weight of 0.10, and weighted end gaps.

Another example of a useful algorithm is the BLAST algorithm, described in: Altschul et al., J. Mol. Biol. 215, 403410, (1990); Altschul et al., Nucleic Acids Res. 25:3389-3402 (1997); and Karlin et al., Proc. Natl. Acad. Sci. U.S.A. 90:5873-5787 (1993). A particularly useful BLAST program is the WU-BLAST-2 program which was obtained from Altschul et al., Methods in Enzymology, 266:460480 (1996); http://blast.wustl/edu/blast/README.html]. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucl. Acids Res., 25:3389-3402. Gapped BLAST uses BLOSUM-62 substitution scores; threshold T parameter set to 9; the two-hit method to trigger ungapped extensions; charges gap lengths of k a cost of 10+k; Xᵤ set to 16, and X_{g} set to 40 for database search stage and to 67 for the output stage of the algorithms. Gapped alignments are triggered by a score corresponding to ˜22 bits.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, “percent (%) nucleic acid sequence identity” with respect to the coding sequence of the polypeptides identified herein is defined as the percentage of nucleotide residues in a candidate sequence that are identical with the nucleotide residues in the coding sequence of the target protein. A preferred method utilizes the BLASTN module of WU-BLAST-2 set to the default parameters, with overlap span and overlap fraction set to 1 and 0.125, respectively.

The alignment may include the introduction of gaps in the sequences to be aligned. In addition, for sequences which contain either more or fewer amino acids than the target protein, it is understood that in one embodiment, the percentage of sequence identity will be determined based on the number of identical amino acids in relation to the total number of amino acids. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

Thus, the variant proteins of the present invention may be shorter or longer than the target protein. Included within the definition of variant proteins are portions or fragments of the target sequence. Fragments of variant proteins are considered variant a proteins if they share a) at least one antigenic epitope; b) have at least the indicated homology; c) and preferably exhibit the biological activity of the target protein.

In a preferred embodiment, as is more fully outlined below, the candidate variant proteins include further amino acid variations, as compared to a target protein, than those outlined herein. In addition, as outlined herein, any of the variations depicted herein may be combined in any way to form additional novel variant proteins.

In addition, candidate variant proteins can be made that are longer than the target protein, for example, by the addition of other sequences, such as purification tags, fusion sequences, etc, as described in U.S. Ser. No. 09/798,789, incorporated herein by reference in its entirety. For example, the variant proteins of the invention may be fused to other therapeutic proteins or to other proteins such as Fc or serum albumin for pharmacokinetic purposes. See for example U.S. Pat. No. 5,766,883 and 5,876,969, both of which are expressly incorporated by reference.

Also included within the invention are variant proteins comprising variable residues in core, surface, and boundary residues.

In a preferred embodiment, the variant proteins of the invention are human conformers. By “conformer” herein is meant a protein that has a protein backbone 3D structure that is virtually the same but has significant differences in the amino acid side chains. That is, the variant proteins of the invention define a conformer set, wherein all of the proteins of the set share a backbone structure and yet have sequences that differ by at least 1-3-5%. The three-dimensional backbone structure of a variant protein thus substantially corresponds to the three dimensional backbone structure of human target protein.

“Backbone” in this context means the non-side chain atoms: the nitrogen, carbonyl carbon and oxygen, and the α-carbon, and the hydrogens attached to the nitrogen and α-carbon. To be considered a conformer, a protein must have backbone atoms that are no more than 2 Å from the human target protein structure, with no more than 1.5 Å being preferred, and no more than 1 Å being particularly preferred. In general, these distances may be determined in two ways. In one embodiment, each potential conformer is crystallized and its three dimensional structure determined. Alternatively, as the former is technically challenging, the sequence of each potential conformer is run in the PDA™ program to determine whether it is a conformer.

Candidate variant proteins may also be identified as being encoded by candidate variant nucleic acids. In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence, with lower homology being preferred.

In a preferred embodiment, a candidate variant nucleic acid encodes a candidate variant protein. As will be appreciated by those in the art, due to the degeneracy of the genetic code, an extremely large number of nucleic acids may be made, all of which encode the variant proteins of the present invention. Thus, having identified a particular amino acid sequence, those skilled in the art could make any number of different nucleic acids, by simply modifying the sequence of one or more codons in a way that does not change the amino acid sequence of the variant protein.

In one embodiment, the nucleic acid homology is determined through hybridization studies. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength and pH. The Tₘ is the temperature (under defined ionic strength, pH and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

The candidate variant proteins and nucleic acids of the present invention are recombinant. As used herein, “nucleic acid” may refer to either DNA or RNA, or molecules that contain both deoxy- and ribonucleotides. The nucleic acids include genomic DNA, cDNA and oligonucleotides including sense and anti-sense nucleic acids. Such nucleic acids may also contain modifications in the ribose-phosphate backbone to increase stability and half-life of such molecules in physiological environments.

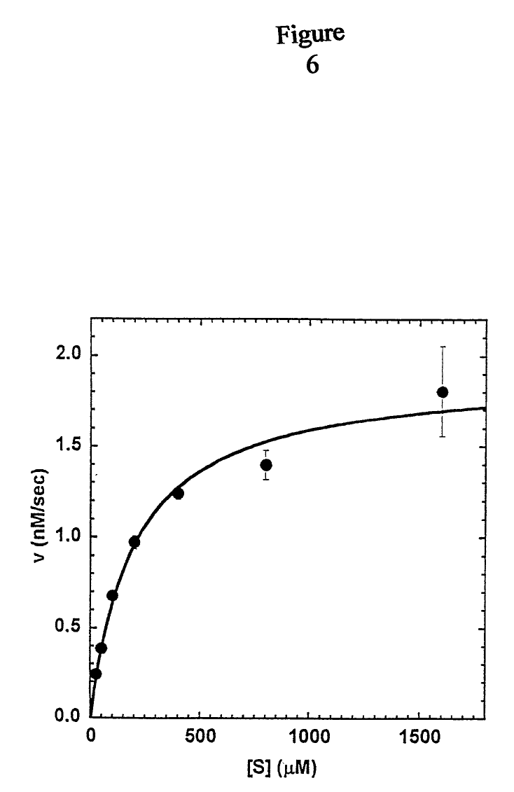

The nucleic acid may be double stranded, single stranded, or contain portions of both double stranded or single stranded sequence. As will be appreciated by those in the art, the depiction of a single strand (“Watson”) also defines the sequence of the other strand (“Crick”); thus the sequence depicted in FIG. 6 also includes the complement of the sequence. By the term “recombinant nucleic acid” herein is meant nucleic acid, originally formed in vitro, in general, by the manipulation of nucleic acid by endonucleases, in a form not normally found in nature. Thus an isolated candidate variant nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a candidate variant protein from one organism in a different organism or host cell. Alternatively, the protein may be made at a significantly higher concentration than is normally seen, through the use of a inducible promoter or high expression promoter, such that the protein is made at increased concentration levels. Furthermore, all of the variant proteins outlined herein are in a form not normally found in nature, as they contain amino acid substitutions, insertions and deletions, with substitutions being preferred, as discussed below.

Also included within the definition of candidate variant proteins of the present invention are amino acid sequence variants of the candidate variant sequences outlined herein. That is, the candidate variant proteins may contain additional variable positions as compared to the target protein. These variants fall into one or more of three classes: substitutional, insertional or deletional variants. These variants ordinarily are prepared by site specific mutagenesis of nucleotides in the DNA encoding a candidate variant protein, using cassette or PCR mutagenesis or other techniques well known in the art, to produce DNA encoding the variant, and thereafter expressing the DNA in recombinant cell culture as outlined above. However, candidate variant protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the candidate variant protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed variant proteins screened for the optimal combination of desired activity. Techniques for making substitution mutations at predetermined sites in DNA having a known sequence are well known, for example, M13 primer mutagenesis and PCR mutagenesis.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the variant protein are desired, substitutions are generally made in accordance with Chart 1:

Chart I

| Original Residue | Exemplary Substitutions |
|---|---|
| Ala | Ser |
| Arg | Lys |
| Asn | Gln, His |
| Asp | Glu |
| Cys | Ser, Ala |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

In some embodiments, it is desirable to have candidate variant proteins with enzyme-like activity that are more stable than the scaffold protein or the wild-type enzyme. Preferably, it would be desirable to have proteins that exhibit oxidative stability, alkaline stability, and thermal stability.

A change in oxidative stability is evidenced by at least about 20%, more preferably at least about 50% increase of activity of a variant protein when exposed to various oxidizing conditions as compared to that of wild-type protein. Oxidative stability is measured by known procedures.

A change in alkaline stability is evidenced by at least about a 5% or greater increase or decrease (preferably increase) in the half life of the activity of a variant protein when exposed to increasing or decreasing pH conditions as compared to that of wild-type protein. Generally, alkaline stability is measured by known procedures.

A change in thermal stability is evidenced by at least about a 5% or greater increase or decrease (preferably increase) in the half-life of the activity of a variant protein when exposed to a relatively high temperature and neutral pH as compared to that of wild-type protein. Generally, thermal stability is measured by known procedures.

The candidate variant proteins and nucleic acids of the invention can be made in a number of ways. Individual nucleic acids and proteins can be made as known in the art and outlined below. Alternatively, libraries of candidate variant proteins can be made for testing.

In a preferred embodiment secondary libraries are generated from primary libraries. As outlined herein, there are a number of different ways to generate a secondary library.

In a preferred embodiment, the primary library of the scaffold protein is used to generate a secondary library. As will be appreciated by those in the art, the secondary library can be either a subset of the primary library, or contain new library members, i.e. sequences that are not found in the primary library. That is, in general, the variant positions and/or amino acid residues in the variant positions can be recombined in any number of ways to form a new library that exploits the sequence variations found in the primary library. That is, having identified “hot spots” or important variant positions and/or residues, these positions can be recombined in novel ways to generate novel sequences to form a secondary library. Thus, in a preferred embodiment, the secondary library comprises at least one member sequence that is not found in the primary library, and preferably a plurality of such sequences.

In one embodiment, all or a portion of the primary library serves as the secondary library. That is, a cutoff is applied to the primary sequences and these sequences serve as the secondary library, without further manipulation or recombination. The library members can be made as outlined below, e.g. by direct synthesis or by constructing the nucleic acids encoding the library members, expressing them in a suitable host, optionally followed by screening.

In a preferred embodiment, the secondary library is generated by tabulating the amino acid positions that vary from a reference sequence. The reference sequence can be arbitrarily selected, or preferably is chosen either as the wild-type sequence or the global optimum sequence, with the latter being preferred. That is, each amino acid position that varies in the primary library is tabulated. Of course, if the original computational analysis fixed some positions, the variable positions of the secondary library will comprise either just these original variable positions or some subset of these original variable positions. That is, assuming a protein of 100 amino acids, the original computational screen can allow all 100 positions to be varied. However, due to the cutoff in the primary library, only 25 positions may vary. Alternatively, assuming the same 100 amino acid protein, the original computational screen could have varied only 25 positions, keeping the other 75 fixed; this could result in only 12 of the 25 being varied in the cutoff primary library. These primary library positions can then be recombined to form a secondary library, wherein all possible combinations of these variable positions form the secondary library. It should be noted that the non-variable positions are set to the reference sequence positions.

The formation of the secondary library using this method may be done in two general ways; either all variable positions are allowed to be any amino acid, or subsets of amino acids are allowed for each position.

In a preferred embodiment, all amino acid residues are allowed at each variable position identified in the primary library. That is, once the variable positions are identified, a secondary library comprising every combination of every amino acid at each variable position is made.

In a preferred embodiment, subsets of amino acids are chosen. The subset at any position may be either chosen by the user, or may be a collection of the amino acid residues generated in the primary screen. That is, assuming core residue 25 is variable and the primary screen gives 5 different possible amino acids for this position, the user may chose the set of good core residues outlined above (e.g. hydrophobic residues), or the user may build the set by choosing the 5 different amino acids generated in the primary screen. Alternatively, combinations of these techniques may be used, wherein the set of identified residues is manually expanded. For example, in some embodiments, fewer than the number of amino acid residues is chosen; for example, only three of the five may be chosen. Alternatively, the set is manually expanded; for example, if the computation picks two different hydrophobic residues, additional choices may be added. Similarly, the set may be biased, for example either towards or away from the wild-type sequence, or towards or away from known domains, etc.

In addition, this may be done by analyzing the primary library to determine which amino acid positions in the scaffold protein have a high mutational frequency, and which positions have a low mutation frequency. The secondary library can be generated by randomizing the amino acids at the positions that have high numbers of mutations, while keeping constant the positions that do not have mutations above a certain frequency. For example, if the position has less than 20% and more preferably 10% mutations, it may be kept constant as the reference sequence position.

In a preferred embodiment, the secondary library is generated from a probability distribution table. As outlined herein, there are a variety of methods of generating a probability distribution table, including using PDA, sequence alignments, force field calculations such as SCMF calculations, etc. In addition, the probability distribution can be used to generate information entropy scores for each position, as a measure of the mutational frequency observed in the library.

In this embodiment, the frequency of each amino acid residue at each variable position in the list is identified. Frequencies can be thresholded, wherein any variant frequency lower than a cutoff is set to zero. This cutoff is preferably 1%, 2%, 5%, 10% or 20%, with 10% being particularly preferred. These frequencies are then built into the secondary library. That is, as above, these variable positions are collected and all possible combinations are generated, but the amino acid residues that “fill” the secondary library are utilized on a frequency basis. Thus, in a non-frequency based secondary library, a variable position that has 5 possible residues will have 20% of the proteins comprising that variable position with the first possible residue, 20% with the second, etc. However, in a frequency based secondary library, a variable position that has 5 possible residues with frequencies of 10%, 15%, 25%, 30% and 20%, respectively, will have 10% of the proteins comprising that variable position with the first possible residue, 15% of the proteins with the second residue, 25% with the third, etc. As will be appreciated by those in the art, the actual frequency may depend on the method used to actually generate the proteins; for example, exact frequencies may be possible when the proteins are synthesized. However, when the frequency-based primer system outlined below is used, the actual frequencies at each position will vary, as outlined below.

As will be appreciated by those in the art and outlined herein, probability distribution tables can be generated in a variety of ways. In addition to the methods outlined herein, self-consistent mean field (SCMF) methods can be used in the direct generation of probability tables. SCMF is a deterministic computational method that uses a mean field description of rotamer interactions to calculate energies. A probability table generated in this way can be used to create secondary libraries as described herein. SCMF can be used in three ways: the frequencies of amino acids and rotamers for each amino acid are listed at each position; the probabilities are determined directly from SCMF (see Delarue et la. Pac. Symp. Biocomput. 109-21 (1997), expressly incorporated by reference). In addition, highly variable positions and non-variable positions can be identified. Alternatively, another method is used to determine what sequence is jumped to during a search of sequence space; SCMF is used to obtain an accurate energy for that sequence; this energy is then used to rank it and create a rank-ordered list of sequences (similar to a Monte Carlo sequence list). A probability table showing the frequencies of amino acids at each position can then be calculated from this list (Koehl et al., J. Mol. Biol. 239:249 (1994); Koehl et al., Nat. Struc. Biol. 2:163 (1995); Koehl et al., Curr. Opin. Struct. Biol. 6:222 (1996); Koehl et al., J. Mol. Bio. 293:1183 (1999); Koehl et al., J. Mol. Biol. 293:1161 (1999); Lee J. Mol. Biol. 236:918 (1994); and Vasquez Biopolymers 36:53-70 (1995); all of which are expressly incorporated by reference. Similar methods include, but are not limited to, OPLS-AA (Jorgensen, et al., J. Am. Chem. Soc. (1996), v 118, pp 11225-11236; Jorgensen, W. L.; BOSS, Version 4.1; Yale University: New Haven, Conn. (1999)); OPLS (Jorgensen, et al., J. Am. Chem. Soc. (1988), v 110, pp 1657ff; Jorgensen, et al., J. Am. Chem. Soc. (1990), v 112, pp 4768ff); UNRES (United Residue Forcefield; Liwo, et al., Protein Science (1993), v 2, pp1697-1714; Liwo, et al., Protein Science (1993), v 2, pp1715-1731; Liwo, et al., J. Comp. Chem. (1997), v 18, pp849-873; Liwo, et al., J. Comp. Chem. (1997), v 18, pp874-884; Liwo, et al., J. Comp. Chem. (1998), v 19, pp259-276; Forcefield for Protein Structure Prediction (Liwo, et al., Proc. Natl. Acad. Sci. USA (1999), v 96, pp5482-5485); ECEPPI3 (Liwo et al., J Protein Chem 1994 May;13(4):375-80); AMBER 1.1 force field (Weiner, et al., J. Am. Chem. Soc. v106, pp765-784); AMBER 3.0 force field (U. C. Singh et al., Proc. Natl. Acad. Sci. USA. 82:755-759); CHARMM and CHARMM22 (Brooks, et al., J. Comp. Chem. v4, pp 187-217); cvff3.0 (Dauber-Osguthorpe, et al.,(1988) Proteins: Structure, Function and Genetics, v4, pp31-47); cff91 (Maple, et al., J. Comp. Chem. v15, 162-182); also, the DISCOVER (cvff and cff91) and AMBER forcefields are used in the INSIGHT molecular modeling package (Biosym/MSI, San Diego Calif.) and HARMM is used in the QUANTA molecular modeling package (Biosym/MSI, San Diego Calif.).

In addition, as outlined herein, a preferred method of generating a probability distribution table is through the use of sequence alignment programs. In addition, the probability table can be obtained by a combination of sequence alignments and computational approaches. For example, one can add amino acids found in the alignment of homologous sequences to the result of the computation. Preferable one can add the wild type amino acid identity to the probability table if it is not found in the computation.

As will be appreciated, a secondary library created by recombining variable positions and/or residues at the variable position may not be in a rank-ordered list. In some embodiments, the entire list may just be made and tested. Alternatively, in a preferred embodiment, the secondary library is also in the form of a rank ordered list. This may be done for several reasons, including the size of the secondary library is still too big to generate experimentally, or for predictive purposes. This may be done in several ways. In one embodiment, the secondary library is ranked using the scoring functions of PDA to rank the library members. Alternatively, statistical methods could be used. For example, the secondary library may be ranked by frequency score; that is, proteins containing the most of high frequency residues could be ranked higher, etc. This may be done by adding or multiplying the frequency at each variable position to generate a numerical score. Similarly, the secondary library different positions could be weighted and then the proteins scored; for example, those containing certain residues could be arbitrarily ranked.

As outlined herein, secondary libraries can be generated in two general ways. The first is computationally, as above, wherein the primary library is further computationally manipulated, for example by recombining the possible variant positions and/or amino acid residues at each variant position or by recombining portions of the sequences containing one or more variant position. It may

In a preferred embodiment, the different protein members of the secondary library may be chemically synthesized. This is particularly useful when the designed proteins are short, preferably less than 150 amino acids in length, with less than 100 amino acids being preferred, and less than 50 amino acids being particularly preferred, although as is known in the art, longer proteins can be made chemically or enzymatically. See for example Wilken et al, Curr. Opin. Biotechnol. 9:412-26 (1998), hereby expressly incorporated by reference.

In a preferred embodiment, particularly for longer proteins or proteins for which large samples are desired, the secondary library sequences are used to create nucleic acids such as DNA which encode the member sequences and which can then be cloned into host cells, expressed and assayed, if desired. Thus, nucleic acids, and particularly DNA, can be made which encodes each member protein sequence. This is done using well known procedures. The choice of codons, suitable expression vectors and suitable host cells will vary depending on a number of factors, and can be easily optimized as needed.

In a preferred embodiment, the secondary library is done by shuffling the family (e.g. a set of variants); that is, some set of the top sequences (if a rank-ordered list is used) can be shuffled, either with or without error-prone PCR. “Shuffling” in this context means a recombination of related sequences, generally in a random way. It can include “shuffling” as defined and exemplified in U.S. Pat. Nos. 5,830,721; 5,811,238; 5,605,793; 5,837,458 and PCT US/19256, all of which are expressly incorporated by reference in their entirety. This set of sequences can also be an artificial set; for example, from a probability table (for example generated using SCMF) or a Monte Carlo set. Similarly, the “family” can be the top 10 and the bottom 10 sequences, the top 100 sequences, etc. This may also be done using error-prone PCR.

Thus, in a preferred embodiment, in silico shuffling is done using the computational methods described therein. That is, starting with either two libraries or two sequences, random recombinations of the sequences can be generated and evaluated.

In a preferred embodiment, error-prone PCR is done to generate the secondary library. See U.S. Pat. Nos. 5,605,793, 5,811,238, and 5,830,721, all of which are hereby incorporated by reference. This can be done on the optimal sequence or on top members of the library, or some other artificial set of family. In this embodiment, the gene for the optimal sequence found in the computational screen of the primary library can be synthesized. Error prone PCR is then performed on the optimal sequence gene in the presence of oligonucleotides that code for the mutations at the variant positions of the secondary library (bias oligonucleotides). The addition of the oligonucleotides will create a bias favoring the incorporation of the mutations in the secondary library. Alternatively, only oligonucleotides for certain mutations may be used to bias the library.

In a preferred embodiment, gene shuffling with error prone PCR can be performed on the gene for the optimal sequence, in the presence of bias oligonucleotides, to create a DNA sequence library that reflects the proportion of the mutations found in the secondary library. The choice of the bias oligonucleotides can be done in a variety of ways; they can chosen on the basis of their frequency, i.e. oligonucleotides encoding high mutational frequency positions can be used; alternatively, oligonucleotides containing the most variable positions can be used, such that the diversity is increased; if the secondary library is ranked, some number of top scoring positions can be used to generate bias oligonucleotides; random positions may be chosen; a few top scoring and a few low scoring ones may be chosen; etc. What is important is to generate new sequences based on preferred variable positions and sequences.

In a preferred embodiment, a variety of additional steps may be done to one or more secondary libraries; for example, further computational processing can occur, secondary libraries can be recombined, or cutoffs from different secondary libraries can be combined. In a preferred embodiment, a secondary library may be computationally remanipulated to form an additional secondary library (sometimes referred to herein as “tertiary libraries”). For example, any of the secondary library sequences may be chosen for a second round of PDA, by freezing or fixing some or all of the changed positions in the first secondary library. Alternatively, only changes seen in the last probability distribution table are allowed. Alternatively, the stringency of the probability table may be altered, either by increasing or decreasing the cutoff for inclusion. Similarly, the secondary library may be recombined experimentally after the first round; for example, the best gene/genes from the first screen may be taken and gene assembly redone (using techniques outlined below, multiple PCR, error prone PCR, shuffling, etc.). Alternatively, the fragments from one or more good gene(s) to change probabilities at some positions. This biases the search to an area of sequence space found in the first round of computational and experimental screening.

In a preferred embodiment, a tertiary library can be generated from combining secondary libraries. Similarly be combined. Sequences from the top of a library can be combined with sequences from the bottom of the library to more broadly sample sequence space, or only sequences distant from the top of the library can be combined. Primary and/or secondary libraries that analyzed different parts of a protein can be combined to a tertiary library that treats the combined parts of the protein. These combinations can be done to analyze large proteins, especially large multidomain proteins or complete protoesomes.

In a preferred embodiment, a tertiary library can be generated using correlations in the secondary library. That is, a residue at a first variable position may be correlated to a residue at second variable position (or correlated to residues at additional positions as well). For example, two variable positions may sterically or electrostatically interact, such that if the first residue is X, the second residue must be Y. This may be either a positive or negative correlation. This correlation, or “cluster” of residues, may be both detected and used in a variety of ways. (For the generation of correlations, see the earlier cited art).

In addition, primary and secondary libraries can be combined to form new libraries; these can be random combinations or the libraries, combining the “top” sequences, or weighting the combinations (positions or residues from the first library are scored higher than those of the second library). There are a wide variety of experimental techniques that can be used to experimentally generate the libraries of the invention, including, but not limited to, Rachitt-Enchira (http://www.enchira.com/gene_shuffling.htm); error-prone PCR, for example using modified nucleotides; known mutagenesis techniques including the use of multi-cassettes; DNA shuffling (Crameri, et al., Nature 391(6664):288-291. (1998)); heterogeneous DNA samples (US5939250); ITCHY (Ostermeier, et al., Nat Biotechnol 17(12):1205-1209. (1999)); StEP (Zhao, et al., Nat Biotechnol 16(3):258-261. (1998)), GSSM (U.S. Pat. Nos. 6,171,820, 5,965,408); in vivo homologous recombination, ligase assisted gene assembly, end-complementary PCR, profusion (Roberts and Szostak, Proc Natl Acad Sci U S A 94(23):12297-12302. (1997)); yeast/bacteria surface display (Lu, et al., Biotechnology (N Y) 13(4):366-372. (1995); Seed and Aruffo, Proc Natl Acad Sci U S A 84(10):3365-3369. (1987); Boder and Wittrup, Nat Biotechnol 15(6):553-557. (1997)).

Using the nucleic acids of the present invention which encode library members, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the library protein. The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. The transcriptional and translational regulatory nucleic acid will generally be appropriate to the host cell used to express the library protein, as will be appreciated by those in the art; for example, transcriptional and translational regulatory nucleic acid sequences from Bacillus are preferably used to express the library protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences include constitutive and inducible promoter sequences. The promoters may be either naturally occurring promoters, hybrid or synthetic promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors and appropriate selection and screening protocols are well known in the art and are described in e.g., Mansour et al., Cell, 51:503 (1988) and Murray, Gene Transfer and Expression Protocols, Methods in Molecular Biology, Vol. 7 (Clifton: Humana Press, 1991).

In addition, in a preferred embodiment, the expression vector contains a selection gene to allow the selection of transformed host cells containing the expression vector, and particularly in the case of mammalian cells, ensures the stability of the vector, since cells which do not contain the vector will generally die. Selection genes are well known in the art and will vary with the host cell used. By “selection gene” herein is meant any gene which encodes a gene product that confers resistance to a selection agent. Suitable selection agents include, but are not limited to, neomycin (or its analog G418), blasticidin S, histinidol D, bleomycin, puromycin, hygromycin B, and other drugs.

In a preferred embodiment, the expression vector contains a RNA splicing sequence upstream or downstream of the gene to be expressed in order to increase the level of gene expression. See Barret et al., Nucleic Acids Res. 1991; Groos et al., Mol. Cell. Biol. 1987; and Budiman et al., Mol. Cell. Biol. 1988.

A preferred expression vector system is a retroviral vector system such as is generally described in Mann et al., Cell, 33:153-9 (1993); Pear et al., Proc. Natl. Acad. Sci. U.S.A., 90(18):8392-6 (1993); Kitamura et al., Proc. Natl. Acad. Sci. U.S.A., 92:9146-50 (1995); Kinsella et al., Human Gene Therapy, 7:1405-13; Hofmann et al., Proc. Natl. Acad. Sci. U.S.A., 93:5185-90; Choate et al., Human Gene Therapy, 7:2247 (1996); PCT/US97/01019 and PCT/US97/01048, and references cited therein, all of which are hereby expressly incorporated by reference.

The candidate proteins of the present invention are produced by culturing a host cell transformed with nucleic acid, preferably an expression vector, containing nucleic acid encoding an library protein, under the appropriate conditions to induce or cause expression of the library protein. As outlined below, the libraries can be the basis of a variety of display techniques, including, but not limited to, phage and other viral display technologies, yeast, bacterial, and mammalian display technologies. The conditions appropriate for library protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

As will be appreciated by those in the art, the type of cells used in the present invention can vary widely. Basically, a wide variety of appropriate host cells can be used, including yeast, bacteria, archaebacteria, fungi, and insect and animal cells, including mammalian cells. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells. See the ATCC cell line catalog, hereby expressly incorporated by reference. In addition, the expression of the secondary libraries in phage display systems, such as are well known in the art, are particularly preferred, especially when the secondary library comprises random peptides. In one embodiment, the cells may be genetically engineered, that is, contain exogeneous nucleic acid, for example, to contain target molecules.

In a preferred embodiment, the library proteins are expressed in mammalian cells. Any mammalian cells may be used, with mouse, rat, primate and human cells being particularly preferred, although as will be appreciated by those in the art, modifications of the system by pseudotyping allows all eukaryotic cells to be used, preferably higher eukaryotes.

Accordingly, suitable mammalian cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells (for use in screening for differentiation and de-differentiation factors), osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, Cos, etc. See the ATCC cell line catalog, hereby expressly incorporated by reference.

Mammalian expression systems are also known in the art, and include retroviral systems. A mammalian promoter is any DNA sequence capable of binding mammalian RNA polymerase and initiating the downstream (3′) transcription of a coding sequence for library protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, library proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of library protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the library protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E. coli, Streptococcus cremoris, and Streptococcus lividans, among others.

The bacterial expression vectors are transformed into bacterial host cells using techniques well known in the art, such as calcium chloride treatment, electroporation, and others.

In one embodiment, candidate proteins are produced in insect cells. Expression vectors for the transformation of insect cells, and in particular, baculovirus-based expression vectors, are well known in the art and are described e.g., in O'Reilly et al., Baculovirus Expression Vectors: A Laboratory Manual (New York: Oxford University Press, 1994).

In a preferred embodiment, candidate proteins are produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

The library protein may also be made as a fusion protein, using techniques well known in the art. Thus, for example, for the creation of monoclonal antibodies, if the desired epitope is small, the library protein may be fused to a carrier protein to form an immunogen. Alternatively, the library protein may be made as a fusion protein to increase expression, or for other reasons. For example, when the library protein is an library peptide, the nucleic acid encoding the peptide may be linked to other nucleic acid for expression purposes. Similarly, other fusion partners may be used, such as targeting sequences which allow the localization of the library members into a subcellular or extracellular compartment of the cell, rescue sequences or purification tags which allow the purification or isolation of either the library protein or the nucleic acids encoding them; stability sequences, which confer stability or protection from degradation to the library protein or the nucleic acid encoding it, for example resistance to proteolytic degradation, or combinations of these, as well as linker sequences as needed.

Thus, suitable targeting sequences include, but are not limited to, binding sequences capable of causing binding of the expression product to a predetermined molecule or class of molecules while retaining bioactivity of the expression product, (for example by using enzyme inhibitor or substrate sequences to target a class of relevant enzymes); sequences signalling selective degradation, of itself or co-bound proteins; and signal sequences capable of constitutively localizing the candidate expression products to a predetermined cellular locale, including a) subcellular locations such as the Golgi, endoplasmic reticulum, nucleus, nucleoli, nuclear membrane, mitochondria, chloroplast, secretory vesicles, lysosome, and cellular membrane; and b) extracellular locations via a secretory signal. Particularly preferred is localization to either subcellular locations or to the outside of the cell via secretion.

In a preferred embodiment, the library member comprises a rescue sequence. A rescue sequence is a sequence which may be used to purify or isolate either the candidate agent or the nucleic acid encoding it. Thus, for example, peptide rescue sequences include purification sequences such as the His₆ tag for use with Ni affinity columns and epitope tags for detection, immunoprecipitation or FACS (fluoroscence-activated cell sorting). Suitable epitope tags include myc (for use with the commercially available 9E10 antibody), the BSP biotinylation target sequence of the bacterial enzyme BirA, flu tags, lacZ, and GST.

Alternatively, the rescue sequence may be a unique oligonucleotide sequence which serves as a probe target site to allow the quick and easy isolation of the retroviral construct, via PCR, related techniques, or hybridization.

In a preferred embodiment, the fusion partner is a stability sequence to confer stability to the library member or the nucleic acid encoding it. Thus, for example, peptides may be stabilized by the incorporation of glycines after the initiation methionine (MG or MGGO), for protection of the peptide to ubiquitination as per Varshavsky's N-End Rule, thus conferring long half-life in the cytoplasm. Similarly, two prolines at the C-terminus impart peptides that are largely resistant to carboxypeptidase action. The presence of two glycines prior to the prolines impart both flexibility and prevent structure initiating events in the di-proline to be propagated into the candidate peptide structure. Thus, preferred stability sequences are as follows: MG(X)nGGPP, where X is any amino acid and n is an integer of at least four.

In one embodiment, the library nucleic acids, proteins and antibodies of the invention are labeled. By “labeled” herein is meant that nucleic acids, proteins and antibodies of the invention have at least one element, isotope or chemical compound attached to enable the detection of nucleic acids, proteins and antibodies of the invention. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

In a preferred embodiment, the library protein is purified or isolated after expression. Library proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the library protein may be purified using a standard anti-library antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the library protein. In some instances no purification will be necessary.

Once synthesized, expressed, and purified if necessary, the candidate proteins and nucleic acids are useful in a number of applications.

Once made, the candidate proteins are tested for enzyme-like activity. These screens will be based on the active site domain chosen. Thus, any number of enzymatic activities or attributes may be tested, including substrate binding, substrate specificity, kinetic properties, such as Kₘ, K_{cat}, etc., assays for determining competitive versus non competitive inhibitors, stability profiles (pH, thermal, buffer conditions), mass spectrometry analysis of intermediates, etc. See also Fersht, A., Enzyme structure and mechanism (Freeman, New York, 1985); Walsh, C. Enzymatic Reaction Mechanisms, (W.H. Freeman and Co., New York, 1979); both of which are expressly incorporated herein by reference).

Candidate proteins with novel enzyme-like activity find use in a wide variety of applications, as will be appreciated by those in the art, ranging from industrial to pharmacological uses, depending on the enzymatic activity. Thus, for example, enzymes exhibiting increased thermal stability may be used in industrial processes that are frequently run at elevated temperatures, for example carbohydrate processing (including saccharification and liquifaction of starch to produce high fructose corn syrup and other sweetners), protein processing (for example the use of proteases in laundry detergents, food processing, feed stock processing, baking, etc.), etc. Similarly, the methods of the present invention allow the generation of useful pharmaceutical proteins, such as analogs of known proteinaceous drugs which are more thermostable, less proteolytically sensitive, or contain other desirable changes, dominant negative inhibitors that find in the removal of toxic substances from the body. Toxic substances include toxins produced by micoorganisms, man-made toxins, as well as naturally made compounds that when present in high levels leads to a disease state.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

Generation of a Thioredoxin Expressing a Novel Catalytic Property: Histidine Mediated Nucleophilic Hydrolysis of p-nitrophenol acetate.

Computational Approach

The thioredoxin protein from E. coli (Holmgren, A., (1985) Annu Rev Biochem, 54: 237-271) was selected as a scaffold due to its favorable expression properties, thermodynamic stability (Broo, K. S., et al., (1998) Fold Des, 3:303-312), and because it is essentially catalytically inert with respect to p-nitrophenol acetate (PNPA) binding and hydrolysis. An active site scan was performed in order to identify favorable positions for the catalytic histidine as well as mutations necessary for substrate recognition and binding. In separate calculations using the ORBIT computational program, each position in the protein structure of thioredoxin was modeled using a set of side chain rotamers for the high energy state of histidine (FIG. 2B). This strategy computationally limits the search to the relevant phase space where histidine and the substrate are properly positioned to undergo chemistry. All other positions in the protein backbone were allowed to chose, with proper consideration for rotamer flexibility, between their wild type identity and alanine in order to accommodate the substrate and to build the active site. After computing the optimal solution using algorithms based on DEE, positions that changed to alanine can be subsequently allowed to change identity to other amino acids in order to form better interactions with the high energy state rotamer.

For all 94 non-glycine, non-proline positions, a combinatorial complexity of approximately 10²⁶ amino acid sequence that corresponded to 10¹⁰¹ rotamer sequences were scanned in approximately two days on 14 195 MHz R10000 processors (Silicon Graphics) running in parallel. Candidate variant protein sequences with the catalytic histidine at different positions in thioredoxin were ranked based on recognition of the high energy state rotamer as well as substrate accessibility to the designed active site (Table 1).

Computational Methodology

For the histidine-PNPA high energy state rotamers, a backbone independent rotamer library, was generated that included nucleophilic attack by both the Nδ and Nε atoms of histidine and attack on both enantiotopic faces of PNPA. The _{X}1 and _{X}2 dihedral angles were based on histidine dihedral angles in a survey of protein structures (Dunbrack, R. L. & Karplus, M., (1993) J Mol Biol, 230: 543-574) and were expanded by ±1 standard deviation from the reported values. Bond lengths and angles as well as additional dihedral angles were optimized using the DREIDING force field (Mayo, S. L., et al., (1990) J Phys Chem, 94: 8897-8909). All other side chains were modeled using a backbone dependent library (Dunbrack, R. L. & Karplus, M., (1993) J Mol Biol, 230: 543-574; incorporated herein by reference) that was expanded about _{X}1 and _{X}2 dihedral angles for aromatic residues, expanded about _{X}1 dihedral angles for aliphatic residues, and unexpanded for polar residues as described in (Dahiyat, B. I., et al., (1997) J Mol Biol, 273:789-796). The calculations used an energy function based on the DREIDING force field. A solvation potential (Dahiyat, B. I., et al., (1997) J Mol Biol, 273:789-796; and Street, A. G. & Mayo, S. L. (1998) Fold Des, 3: 253-258) was selectively applied tot he high energy state rotamers in order to favor substrate recognition. The solvent accessible surface area (Lee, B. & Richards, F. M. (1971) J Mol Biol, 55:379-400) based potential calculated with the Connolly algorithm (Connolly, M. L. (1983) Science, 231: 709-713) included a hydrophobic burial benefit of 48 cal/mol/Ω² and a hydrophobic exposure penalty of 76.8 cal/mol/Ω² as previously described (38,41). A Lennard-Jones 12-6 potential with van der Waals radii scaled by 0.9 was applied to all atomic interactions (Dahiyat & Mayo (1997) Proc Natl Acad Sci USA, 94:10172-10177). All residues, including the high energy state complex, with hydrogen bond donors or acceptors used a distance and angle dependent hydrogen bond potential as well as a simple electrostatic potential as previously described (Dahiyat, et al., (1997) Protein Sci, 6:1333-1337).

The hydrophobic solvent accessible surface area of substrate atoms computed using the Lee and Richards definition (Lee, B. & Richards, F. M. (1971) J Mol Biol, 55:379-400) and the Connolly algorithm (Connolly, M. L. (1983) Science, 231: 709-713) was used to evaluate recognition. Total solvent accessible surface area of the high energy state rotamer was used to evaluate substrate accessibility. Computed designs with zero total solvent accessible surface area for the substrate were considered inaccessible to substrate and were eliminated from further consideration.

Experimental Validation

The top two sequences from the limited combinatorial complexity active site scan were selected for experimental analysis. Protozyme design 1 (PZD1) contains two mutations required to introduce the catalytic histidine and to build the active site (F12H and Y70A), while PZD2 contains three mutations (F12A, L17H, and Y70A).

Experimental Methods:

The background mutation D261 was included in both PZD1 and PZD2. D261 was predicted by ORBIT in an independent calculation and results in increased thermodynamic stability similar to the previously reported D26A protein (Gleason, F. K. (1992) Protein Sci, 1:609-616). Position 26 is distal in space to the designed active sites in both PZD1 and PZD2.

The genes for PZD1 and PZD2 were constructed by site directed mutagenesis using the wild type thioredoxin gene (Invitrogen) cloned into PET-11A. Protein expression was induced with 0.5 mM IPTG from BL21 (DE3) cells grown to mid log phase. Cells were lysed by sonication and pelleted twice at 20,000 g for 30 minutes. The soluble fraction was brought to 60% acetronitrile, pelleted twice and rotory evaporated to half volume. For initial studies, purification was accomplished by reveres phase high performance liquid chromatography. For subsequent studies with PZD2, all protein samples were additionally purified by ion exchange and size exclusion chromatography to a purity of >99% as judged by silver stained PAGE.

PZD2 was dialyzed extensively against 10 mM sodium phosphate buffer at pH 6.95. Kinetic experiments at 22° C. were started by the addition of substrate dissolved in acetonitrile to buffer solution with and without PZD2 (final protein concentration of 4 μM). Protein concentration was determined by UV absorbance in 6M guanidinium hydrochloride assuming an extinction coefficient of 12400 M⁻¹cm⁻¹ at 280 nm. Product concentration was determined by the change in absorbance at 400.5 nm assuming an extinction coefficient for deprotonated PNP of 19700 M^{31 1}cm⁻¹. Final acetronitrile concentration was 1% for all experiments. The steady state rate of hydrolysis by PZD2 was corrected for the buffer rate. Burst phase hydrolysis assays were performed at a substrate concentration of 1.6 mM. Protein concentration was 4 μM, with the exception of wild type thioredoxin. Wild type thioredoxin was assayed at 50 μM to improve signal to noise and extrapolated to a protein concentration of 4 μM. The dead time for this experiment was approximately 30 seconds.

The +42 mass unit species detected in the trapping experiment is a result of replacement of a hydrogen (−1) with an acetyl group (+43). 100 μM PZD2 in 10 mM TRIS at pH 7.0 was reacted with 1.6 mM PNPA to steady state conditions and a mass spectrum acquired. The same protein solution without PNPA was used as a control. Burst phase kinetics in this buffer system yielded essentially identical results as in phosphate buffer.

Experimental Results:

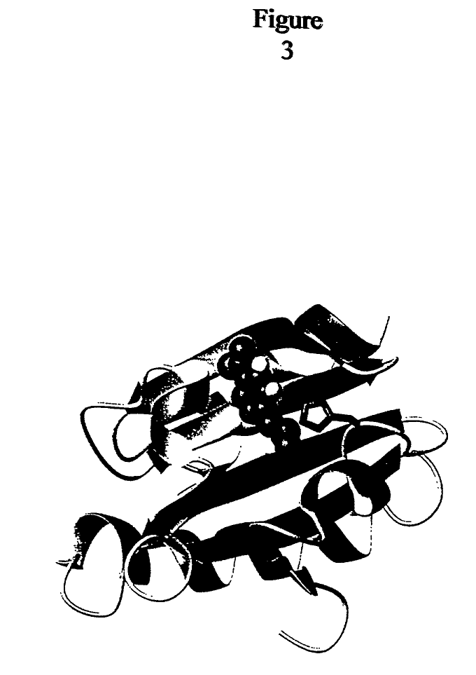

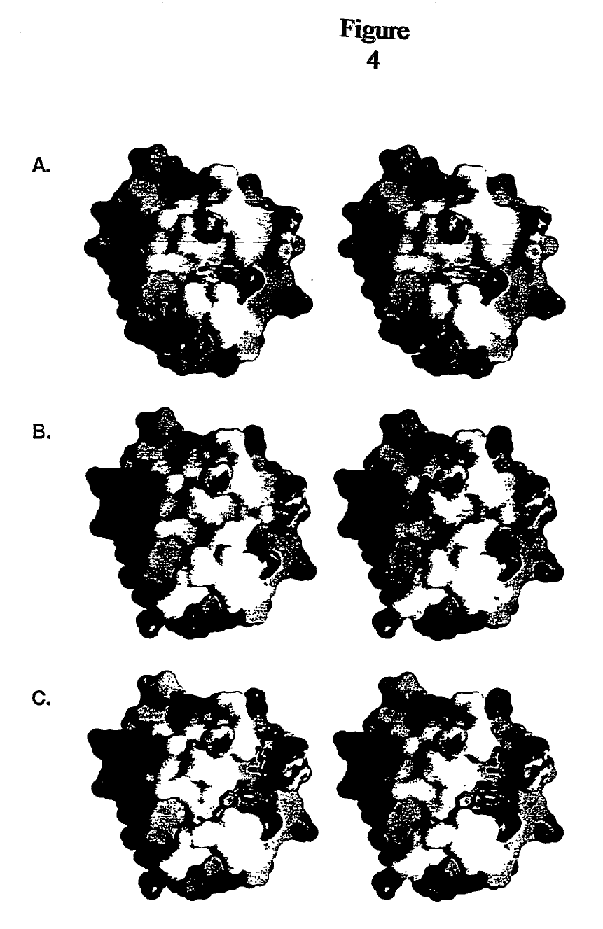

Experimentally both proteins demonstrated catalytic hydrolysis of PNPA at a rate significantly above background. Based on preliminary kinetic experiments, PZD2 was selected for further analysis. The designed structure of PZD2 computed by ORBIT shows the substrate atoms in a cleft created above the β sheet and between two a helices (FIG. 3). This cleft (FIG. 4A) is not present in the wild type thioredoxin x-ray structure (Katti, S. K., et al., (199) J Mol Bio, 212:167-184; FIG. 4B), indicating that the space creating mutations (F12A and Y70A) are necessary to create the putative substrate binding site (FIG. 4C).

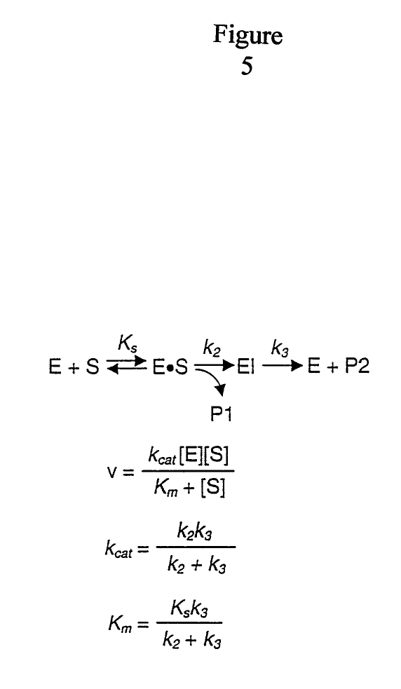

The rate of PNPA hydrolysis by PZD2 was experimentally determined over a range of substrate concentrations using standard Briggs-Haldane steady state treatment (FIG. 5). The reaction velocity of PZD2 demonstrated saturation kinetics with respect to increasing substrate concentration (FIG. 6), indicating that the molecule is acting as an enzyme-like protein with both substrate saturation and catalytic rate enhancement. Hofstee analysis of the data gives a Kₘ of 170±20 μM, K_{cat} of 4.6±0.2×10⁻⁴ sec⁻¹, and K_{cat}/K_{uncat} of 180. The kinetics of PZD2 are comparable to those of the first catalytic antibodies: Kₘ of 208 μM and K_{cat}/K_{uncat} of 770 for MOPC67 (Pollack, S. J., et al., (1986) Science, 234: 1570-1573) and Kₘ of 1.9 μM and K_{cat/K}_{uncat} of 960 for 6D4 (Tramontano, A., (1986) Science, 234:1566-1570).

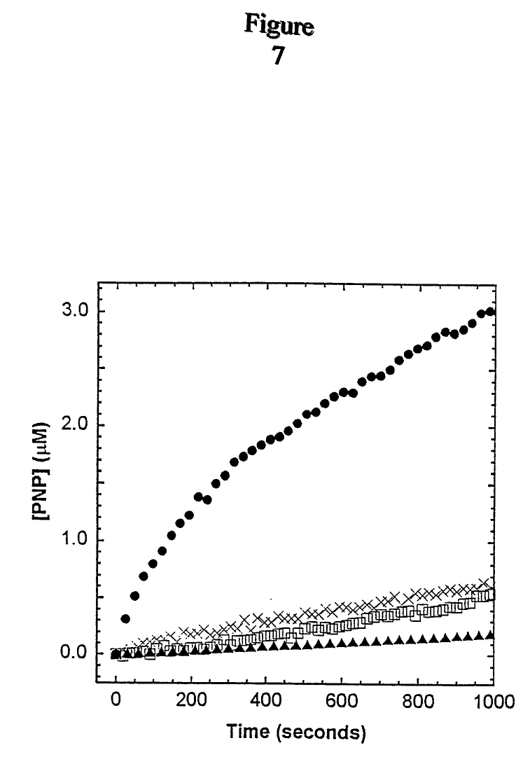

At high substrate concentrations, kinetics with an initial “burst” phase are a common feature of nutural enzymes and are a consequence of a kinetic bottleneck on the reaction pathway. For nucleophilic hydrolysis of PNPA, the rate of PNP formation should decrease and plateau as the acyl-enzyme intermediate concentration approaches a steady state. PZD2 displays burst phase kinetics at high substrate concentration (FIG. 7) consistent with the formation of an enzyme intermediate. The burst phase kinetics could also be a result of slow product release, however this is unlikely given the kinetic parameters of PZD2. Based on the burst phase data, we estimate a Kₛ=1 mM, K₂=3 3×10⁻³ sec⁻¹, and K₃=8×10⁻⁴ sec⁻¹.

Wild type thioredoxin is essentially inactive, but does show weak second order PNPA hydrolysis consistent with its single surface exposed histidine at position 6. Mutation of the designed catalytic histidine to alanine in PZD2 (HI 7A) results in a protein with catalytic activity similar to wild type thioredoxin indicating that the designed catalytic histidine at position 17 is necessary for the enzyme-like activity in PZD2 (FIG. 7). Mutation of the two other active site residues in PZD2 back to their wild type identities (A12F and A70Y) also results in a protein with activity similar to wild type. Additionally, at pH 5.7 the activity of PZD2 is almost entirely eliminated, consistent with protonation of the catalytic histidine. The kinetic and mutational evidence strongly indicate that PZD2 is working as designed with H17 acting as a catalytic nucleophile and the space creating mutation (F12A and Y70A) forming a binding site for the substrate.

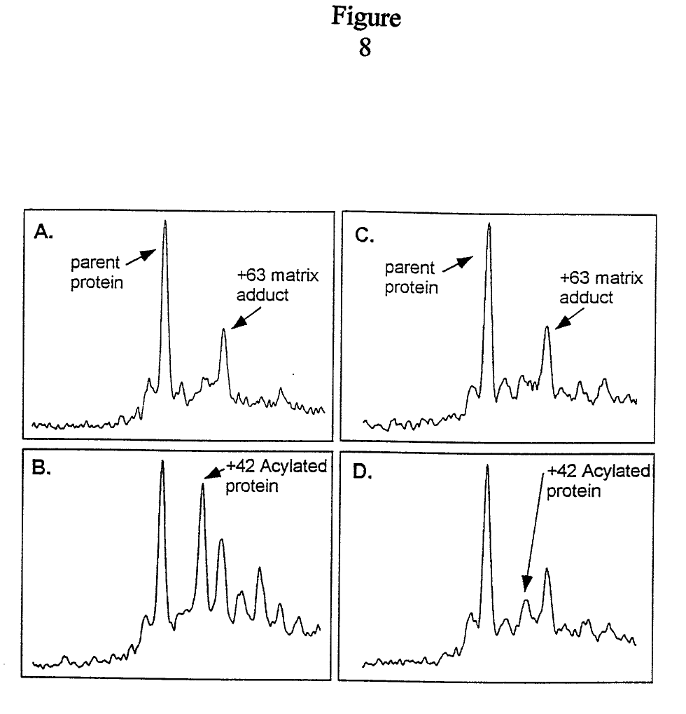

Reaction of PZD2 with PNPA should yield an acyl-enzyme intermediate, increasing the population of 30 +42 mass unit species compared to free protein. Mass spectrometry of PZD2 clearly indicates increased population of an acylated species upon reaction with PNPA (FIGS. 8A & 8B). Acylation due to reaction with PNPA is essentially absent for the H17A mutant of PZD2 (FIGS. 8C & 8D), again indicating that H17 is the active nucleophile.

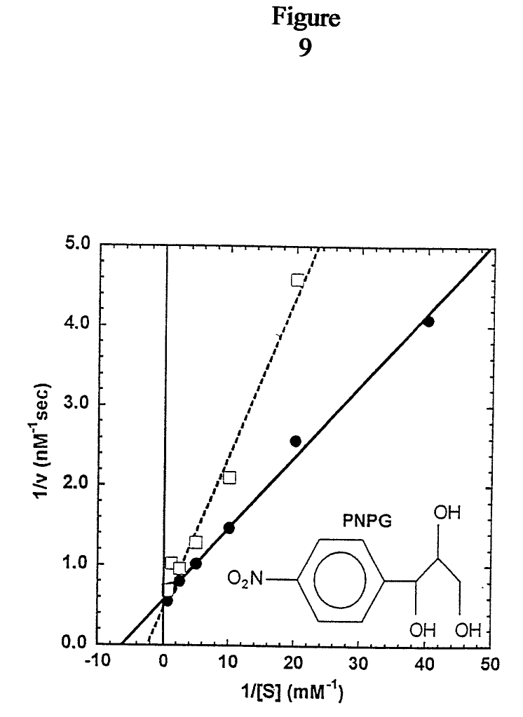

Naturally occurring enzymes are frequently inhibited in a competitive manner by inert compounds resembling the substrate molecule. With this in mind, we assayed the inhibitory effects of p-nitrophenol glycerol (PNPG), a highly soluble compound with structural similarity to PNPA. A double reciprocal analysis of PZD2 catalyzed PNPA hydrolysis in the presence and absence of inhibitor shows the hallmark features of competitive inhibition (FIG. 9) implying that PNPG is able to bind in the active site and block PNPA binding. A Dixon analysis of PNPG inhibition at fixed substrate concentration yields a K₁ of 20 mM. The decreased binding affinity of PNPG relative to PNPA may be due to burial of polar hydroxyl groups against hydrophobic regions in the active site or differences in the steric requirements for PNPG and PNPA. p-nitrophenyl phosphate was also tested, but it did not show detectable inhibition at concentrations up to 20 mM, suggesting that PZD2 binds preferentially to uncharged nitophenyl molecules.

In order to further test the substrate specificity of PZD2 we assayed its kinetics with p-nitrophenyl propionate (PNPP) which has an additional methylene group compared to PNPA. Hofstee analysis indicates within error an identical K_{cat}/K_{uncat} and a Kₘ of 110±20 μM for PNPP, compared to 170±20 μM for PNPA. Inspection of the designed structure of PZD2 with bound PNPA shows that space exists to accommodate the additional methylene of PNPP. Both favorable van der Waals interactions and hydrophobic burial afforded by the relatively open active site likely explain the slightly increased affinity of PZD2 for PNPP compared to PNPA.

TABLE 1
Top 10 Designs from an Active Site Scan

| | Catalytic | Fraction | Number of | |
|---|---|---|---|---|
| | Histidine | Hydrophobic | Site Active | |
| Design | Position | Exposure | Mutations | Active Site Mutations |
| PZD1 | 12 | 0.11 | 2 | F12H, Y70A |
| PZD2 | 17 | 0.15 | 3 | F12A, L17H, Y70A |
| PZD3 | 86 | 0.29 | 4 | V86H, I38A, L42A, |
| | | | | L99A |
| PZD4 | 72 | 0.34 | 2 | I72H, L79A |
| PZD5 | 66 | 0.34 | 3 | T66H, F12A, Y70A |
| PZD6 | 6 | 0.36 | 0 | None |
| PZD7 | 39 | 0.37 | 2 | A39H, K57A |
| PZD8 | 91 | 0.39 | 2 | V91H, T77A |
| PZD9 | 49 | 0.39 | 2 | Y49H, K52A |
| PZD10 | 77 | 0.43 | 3 | T77H, L79A, T89A |

In Table 1, the designs are ranked based on hydrophobic surface area burial of substrate atoms in the high energy state complex. The top two designs, PZD1 and PZD2 were experimentally tested for catalytic activity as described above. Interestingly, the wild type sequence (PZD6; H at position 6 with no binding site mutations) is present among the top ranked designs. As the wild type protein does not exhibit enzyme-like activity toward PNPA hydrolysis, relatively few active sites designs appear to be accessible within the thioredoxin scaffold. The use of a larger scaffold while requiring additional computational effort would likely result in improved designs.

We claim:

1. A method for generating a protozyme, said method comprising:
a) identifying a suitable protein scaffold lacking a desirable enzyme-like activity;
b) inputting a protein backbone structure of said protein scaffold into a computer, wherein said backbone structure has variable residue positions;
c) inserting an active site domain into said scaffold;
d) applying at least one protein design cycle; and
e) generating a set of candidate variant proteins with putative enzyme-like activity.

2. A method according to claim 1 wherein said insertion step is done at the same time as said protein design cycle.

3. A method according to claim 1 wherein said insertion step is done prior to said applying step.

4. A method according to claim 1 wherein said insertion step is done after said applying step.

5. A method according to claim 1 wherein said insertion step comprises the use of at least one high energy state rotamer.

6. A method according to claim 1 further comprising applying a second protein design cycle prior to said generating step.

7. A method according to claim 1 wherein said active site domain catalyzes a known enzymatic reaction.

8. A method according to claim 1 wherein said active site domain catalyzes an unknown enzymatic reaction.

9. A method according to claim 1 wherein said active site domain is a ligand binding domain.

10. A method according to claim 1 wherein said protein design cycle comprises a DEE computation

11. A method according to claim 1 wherein said protein design cycle includes the use of at least one scoring function.

12. A method according to claim 8 wherein said scoring function is selected from the group consisting of a van der Waals potential scoring function, a hydrogen bond potential scoring function, an atomic salvation scoring function, an electrostatic scoring function and a secondary structure propensity scoring function.

13. A method according to claim 1 further comprising synthesizing a plurality of secondary sequences to generate a library of putative protoenzymes.

14. A method according to claim 13 wherein said synthesis includes a shuffling step.

15. A method according to claim 1 further comprising testing said candidate variant proteins for said catalytic property.

16. A method according to claim 1 wherein said protein design cycle comprises protein design automation.

17. A method according to claim 1 wherein said protein design cycle comprises a force field calculation.
